(19) United States
(12) Patent Application Publication
(10) Pub. No.: US 2024/0317789 A1
(43) Pub. Date: Sep. 26, 2024

(54) PRODRUGS OF TAPINAROF

(71) Applicants: Allianthera (Suzhou) Biopharmaceutical Co., Ltd., Suzhou (CN); Allianthera Boston Inc., Natick, MA (US)

(72) Inventors: Jinshan Chen, Madison, CT (US); Rajiv Chopra, Andover, MA (US)

(21) Appl. No.: 18/580,184

(22) PCT Filed: Aug. 5, 2022
(86) PCT No.: PCT/US22/39499

§ 371 Date: Jan. 18, 2024

Related U.S. Application Data
(60) Provisional application No. 63/230,242, filed on Aug. 6, 2021.

(30) Foreign Application Priority Data
Aug. 6, 2021 (WO) PCT/CN21/111116

(51) Int. Cl. C07H 3/06; A61K 9/00; A61K 31/095; A61K 31/235; A61K 31/351; A61K 31/661; A61P 1/04; C07C 69/40; C07C 271/52; C07C 271/54; C07C 271/58; C07C 305/24; C07C 307/02; C07F 9/09; C07F 9/24

(52) U.S. Cl. CPC C07H 3/06; A61K 9/0014; A61K 9/0053; A61K 31/095; A61K 31/235; A61K 31/351; A61K 31/661; A61P 1/04; C07C 69/40; C07C 271/52; C07C 271/54; C07C 271/58; C07C 305/24; C07C 307/02; C07F 9/093; C07F 9/242

(57) ABSTRACT

Disclosed herein are prodrugs of tapinarof and pharmaceutical formulations comprising tarpinarof, such as oral formulations. Further disclosed herein are methods for treating diseases and disorders of the gastrointestinal tract, skin, eye, lung, and bone joints via the tapinarof prodrugs.

### BACKGROUND OF THE INVENTION

### Field of the Invention

This disclosure relates to prodrugs of tapinarof, pharmaceutical formulations thereof, and methods of using the prodrugs to treat diseases and disorders, such as diseases and disorders of the gastrointestinal tract, skin, lung, eyes, and/or bone joints.

### Description of Related Technology

Tapinarof ((E)-2-isopropyl-5-styrylbenzene-1,3-diol, compound a) is a small-molecule that has been found to be useful as a topical therapeutic for the treatment of psoriasis and atopic dermatitis. Tapinarof is known to bind to and activate biological targets, such as the aryl hydrocarbon receptor (AHR), a ligand-dependent transcription factor that regulates gene expression in a variety of cells, such as epithelial and immune cells. Therefore, the binding of tapinarof to the AHR can lead to changes of the inflammation cytokine profile and antioxidant response in the cellular environment, and can facilitate the healing process of a diseased tissue.

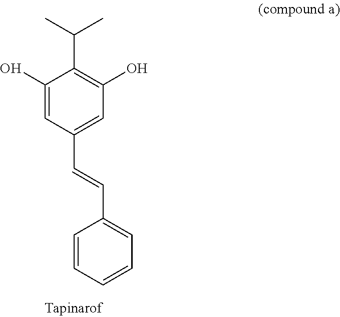 [SMILES: CC(C)c1c(O)cc(/C=C/c2ccccc2)cc1O]

In particular, AHR signaling plays a key role in maintaining skin homeostasis by regulating the skin immune network, keratinocyte differentiation, skin barrier function and pigmentation, and responses to oxidative stress. Cells found in the skin, including keratinocytes, sebocytes, fibroblasts, melanocytes, endothelial cells, Langerhans cells, and other immune cells possess AHR. Accordingly, modulation of the AHR signal transduction pathway is implicated in the pathology of various diseases and disorders of the skin.

AHR signaling also is known to regulate the composition and function of different cell types in the gastrointestinal tract, and therefore, has a key role in maintaining the balance between health and disease. AHR is involved in several physiological processes, including regulation of homeostasis and immunity at epithelial barriers such as the one formed by intestinal epithelial cells (IECs). Potent immune responses in the body occur in the gut and, as such, there is considerable interest in elucidating the molecular mechanism(s) underlying the role of AHR in cells in the intestinal mucosa—including IECs and various immune cells, such as B cells, T cell receptor γδ T cells (TCRγδ), T helper 17 cells (Th17), regulatory T cells (Treg), type 1 regulatory T cells (Tr1), innate lymphoid cells (ILC), macrophages (MQ), intraepithelial lymphocytes (IEL), dendritic cells (DC), and neutrophils. Accordingly, aberrant AHR activity has been implicated in several intestinal pathologies, such as intestinal inflammation, infection and cancer.

AHR signaling also is known to play a key role in bone remodeling by altering the interplay between bone-forming osteoblasts and bone-resorbing osteoclasts. The overall effect of AHR activation in osteoblasts is suppressed cell differentiation, and AHR agonism has dose-dependent effects on osteoblasts in which hyperactivation and hypoactivation, respectively, inhibit and promote bone formation. The effect of AHR modulation in osteoclasts is less well-understood, but the AHR pathway has been implicated in both stimulation and impairment of osteoclast differentiation. Accordingly, the AHR pathway is an attractive target for the treatment of various human diseases in which osteoblasts and osteoclasts are implicated in pathogenesis, including bone destructive diseases such as osteoporosis and cancer.

Systemic exposure of AHR modulators may limit the safety margin of a therapeutic regime. As such, there is a need for novel methods of delivering an AHR modulator (e.g., tapinarof) into a target tissue via a prodrug of the modulator and minimizing systemic exposure of the modulator.

### SUMMARY

One aspect of the disclosure provides a compound having a structure of Formula (I):

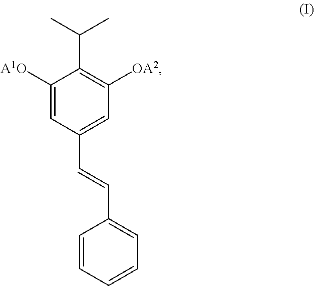

or a pharmaceutically acceptable salt thereof, wherein: each of A¹ and A² independently is H, C₁₋₆alkyl, or

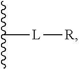

and at least one A¹ and A² is

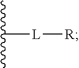

each L independently is a bond,

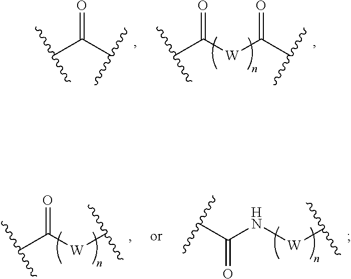

### W is

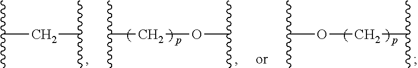

n is an integer from 1-6; p is 2 or 3; and each R independently is a polar group capable of forming at least two hydrogen bonds.

In some embodiments, one of A¹ and A² is H or C₁₋₆alkyl and the other of A¹ and A² is

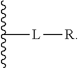

For example, one of A¹ and A² is H or CH₃.

In some cases, each of A¹ and A² is

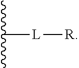

In some embodiments, each R independently is selected from the group consisting of

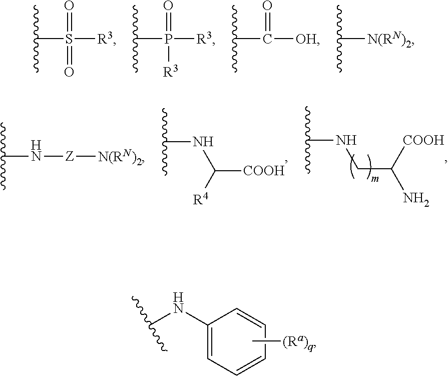

and a saccharide; wherein: m is 1, 2, 3, 4, 5, or 6; q is 2, 3, 4, or 5; Z is C₂₋₆alkylene or C₂₋₆polyoxyalkene; each R^{a} independently is

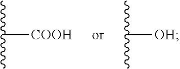

each R^{N} independently is H or CH₃; each R³ independently is OH or NH₂; each R⁴ independently is H, C₁₋₆alkyl, or

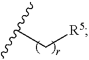

wherein: r is an integer from 1-6; R⁵ is selected from the group consisting of

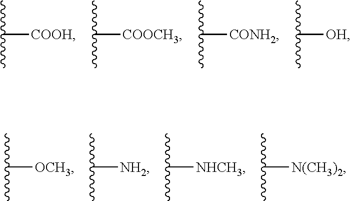

heteroaryl comprising 5 or 6 total ring atoms and 1, 2, or 3 heteroatoms selected from N, O, and S,

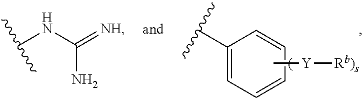

wherein s is 1, 2, 3, 4, or 5; Y is absent or C₁₋₂alkylene; and R^{b} is selected from the group consisting of

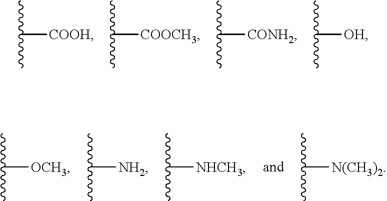

In some cases, at least one L is a bond. In various cases, each L is a bond. In some embodiments, R selected from the group consisting of

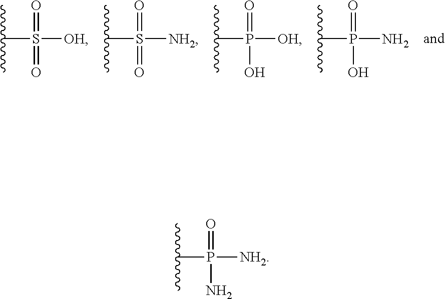

In various embodiments, (i) each R is

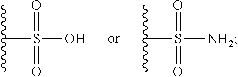 [SMILES: *S(=O)(=O)O ; *S(N)(=O)=O]

or (ii) each R is

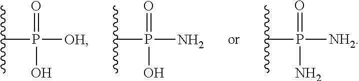

In some cases, (i) each R is

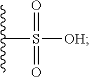 [SMILES: *S(=O)(=O)O]

or (ii) each R is

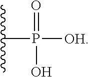 [SMILES: *P(=O)(O)O]

In some embodiments, at least one L is

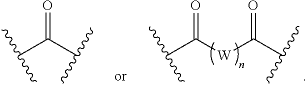

In various embodiments, n is 2, 3, or 4. In some cases, W is

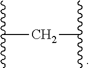 [SMILES: *C*]

In various embodiments, W is

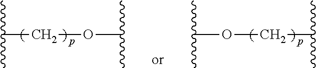

and p is 2. In some embodiments, L is not a bond and each R independently is

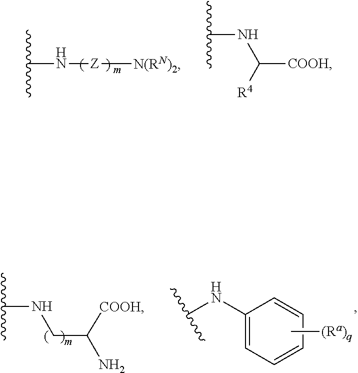

or a saccharide.

In various embodiments, at least one R is

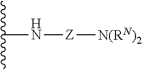

and each R^{N} is H. In some cases, at least one R is

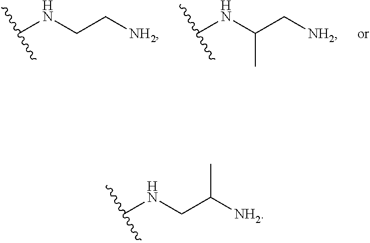

In various embodiments, at least one R is

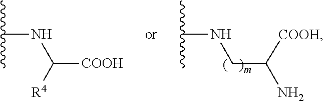

R⁴ is H or CH₃, and m is 1, 2, or 3. In some cases, at least one R is

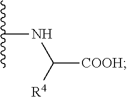

### R⁴ is

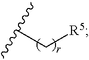

### r is 1, 2, or 3; and R⁵ is

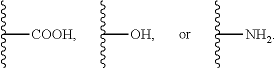

In various cases, at least one R is

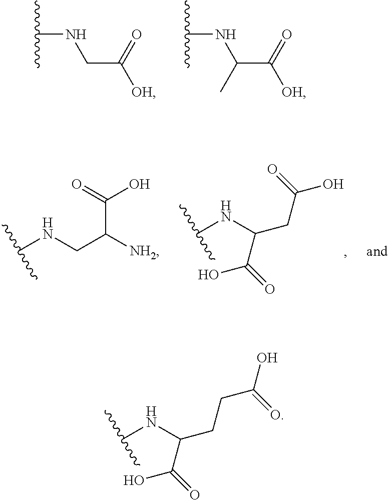

In some embodiments, at least one R is

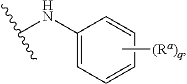

q is 2 or 3; and each R^{a} independently is

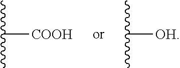

In various embodiments, at least one R is

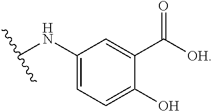 [SMILES: *Nc1ccc(O)c(C(=O)O)c1]

In some cases, at least one R is a saccharide. In various cases, the saccharide is an amino saccharide, a monosaccharide, a disaccharide, or an oligosaccharide. In some embodiments, the saccharide is an oligosaccharide comprising 3-10 monosaccharide units. In various embodiments, the oligosaccharide comprises α-1,4 glycosidic bonds. In some cases, the oligosaccharide is a cyclic oligosaccharide having 5-8 pyranose units. In some embodiments, the pyranose units comprise glucose. In various embodiments, the oligosaccharide comprises a cyclodextrin. In some cases, at least one R is

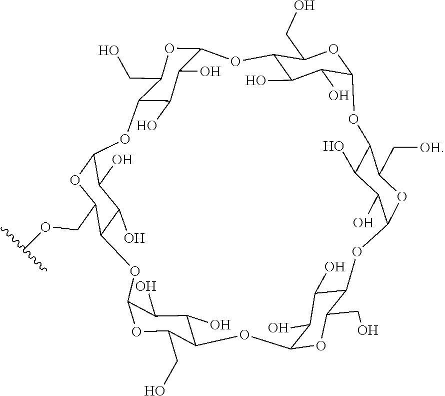 [SMILES: *OCC1OC2OC3C(CO)OC(OC4C(CO)OC(OC5C(CO)OC(OC6C(CO)OC(OC7C(CO)OC(OC1C(O)C2O)C(O)C7O)C(O)C6O)C(O)C5O)C(O)C4O)C(O)C3O]

In some embodiments, at least one L is

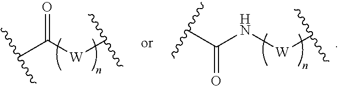

In some embodiments, n is 2, 3, or 4. In some cases, W is

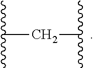 [SMILES: *C*]

In various cases, at least one L is

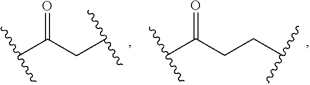 [SMILES: *CC(*)=O ; *CCC(*)=O]

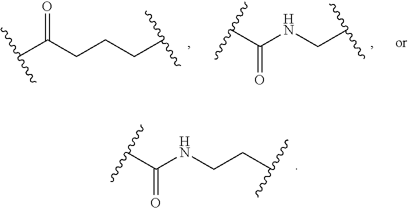 [SMILES: *CCCC(*)=O ; *CNC(*)=O ; *CCNC(*)=O]

In some embodiments, W is

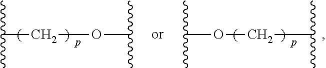

and p is 2. In various embodiments, at least one L is

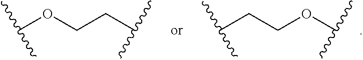 [SMILES: *CCO* ; *CCO*]

In some cases, at least one R is

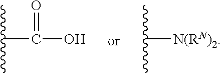

In various cases, at least one R is

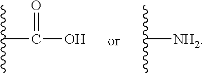

In some embodiments, each R independently is

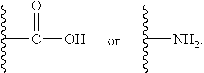

In some embodiments, at least one of A¹ and A² is selected from the group consisting of

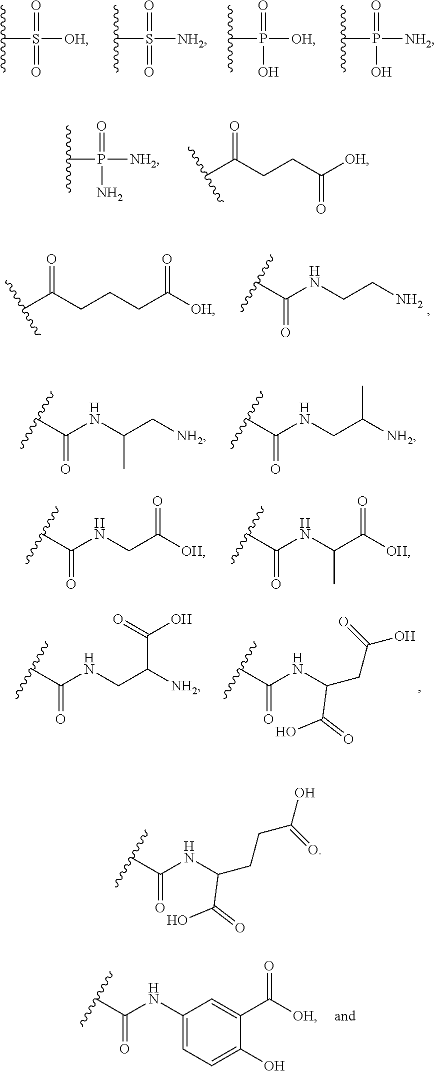

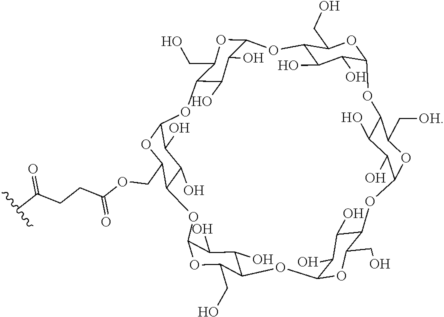 [SMILES: *C(=O)CCC(=O)OCC1OC2OC3C(CO)OC(OC4C(CO)OC(OC5C(CO)OC(OC6C(CO)OC(OC7C(CO)OC(OC1C(O)C2O)C(O)C7O)C(O)C6O)C(O)C5O)C(O)C4O)C(O)C3O]

In some cases, A¹ and A² are the same. In various cases, A¹ and A² are different.

In some embodiments, the compound of Formula (I) is a compound listed in Table 1, or a pharmaceutically acceptable salt thereof. In various embodiments, the compound or salt is optically pure. In some cases, the compound or salt comprises one or more deuterium atoms.

Another aspect of the disclosure provides a pharmaceutical formulation comprising the compound or salt disclosed herein and a pharmaceutically acceptable excipient. In some embodiments, the formulation is as an oral formulation. In various embodiments, the formulation is a topical formulation.

Yet another aspect of the disclosure provides a method of delivering 2-isopropyl-5-(E)-2-phenylethenyl]benzene-1,3-diol (compound a):

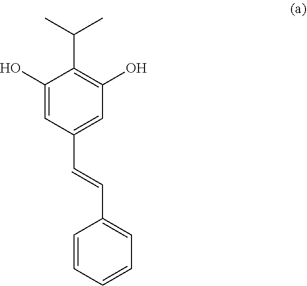 [SMILES: CC(C)c1c(O)cc(/C=C/c2ccccc2)cc1O]

to the intestine (e.g., small intestine and/or large intestine) of a subject comprising administering to the subject a compound, salt, or pharmaceutical formulation disclosed herein. In some embodiments, compound a is released from the prodrug or salt in the intestine (e.g., the small and/or large intestine.

Still another aspect of the disclosure provides a method of modulating the aryl hydrocarbon receptor (AHR) in a cell comprising contacting the cell with a therapeutically effective amount of a compound, salt, or pharmaceutical formulation disclosed herein in an amount effective to modulate the AHR. In some embodiments, the contacting occurs in vivo. In various embodiments, the contacting comprises administering to a subject in need thereof. In some cases, the subject suffers from a disease or disorder of the gastrointestinal tract, skin, or bone joints.

Another aspect of the disclosure provides a method of treating a disease or disorder in a subject, comprising administering to the subject a therapeutically effective amount of a compound, salt, or pharmaceutical formulation. In some embodiments, the disease or disorder is a disease or disorder of the gastrointestinal tract, skin, eye, lung, or bone joints. In various embodiments, the disease or disorder of the gastrointestinal tract is selected from the group consisting of colitis, inflammatory bowel disease, Crohn's disease, celiac disease, necrotizing enterocolitis, irritable bowel syndrome, chronic idiopathic constipation, traveler's diarrhea, and colorectal cancer. In some cases, the disease or disorder of the skin is selected from the group consisting of atopic dermatitis, psoriasis, and vitiligo. In various cases, the disease or disorder of the eye is age-related macular degeneration. In some embodiments, the disease or disorder of the lung is lung fibrosis or chronic obstructive pulmonary disease. In some cases, the disease or disorder of the bone joints is selected from the group consisting of osteoporosis, rheumatoid arthritis, and bone cancer.

Another aspect of the disclosure provides a compound, salt, or pharmaceutical formulation disclosed herein for use in the treatment of a disease or disorder of the gastrointestinal tract, skin, eye, lung or bone joints. The disclosure also provides use of a compound, salt, or pharmaceutical formulation for the manufacture of a medicament for use in the treatment of a disease or disorder of the gastrointestinal tract, skin, eye, lung, or bone joints.

Yet another aspect of the disclosure provides a method of preparing the compound or salt of the disclosure, comprising admixing 2-isopropyl-5-[(E)-2-phenylethenyl]benzene-1,3-diol (compound a):

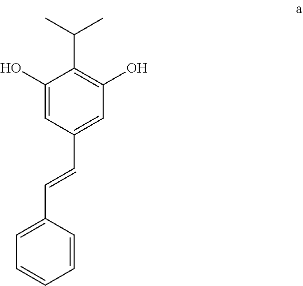 [SMILES: CC(C)c1c(O)cc(/C=C/c2ccccc2)cc1O]

with A¹-LG, A²-LG, or both; wherein LG is a leaving group, to form a compound of Formula (I) or salt thereof.

Still another aspect of the disclosure provides a prodrug of 2-isopropyl-5-[(E)-2-phenylethenyl]benzene-1,3-diol (compound a):

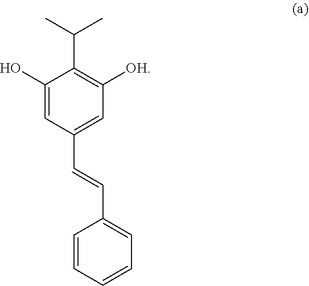 [SMILES: CC(C)c1c(O)cc(/C=C/c2ccccc2)cc1O]

Yet another aspect of the disclosure provides a method of delivering 2-isopropyl-5-(E)-2-phenylethenyl]benzene-1,3-diol (compound a):

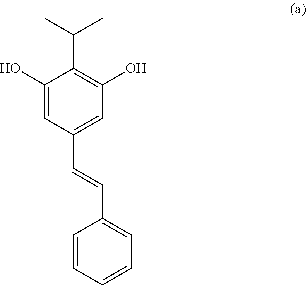 [SMILES: CC(C)c1c(O)cc(/C=C/c2ccccc2)cc1O]

to the intestine (e.g., the small intestine and/or the large intestine) of a subject comprising administering to the subject a prodrug of compound (a), or a pharmaceutically acceptable salt thereof. In some embodiments, compound a is released from the prodrug or salt in the intestine (e.g., the small intestine and/or the large intestine).

Further aspects and advantages will be apparent to those of ordinary skill in the art from a review of the following detailed description, taken in conjunction with the drawings. While the compounds and methods disclosed herein are susceptible of embodiments in various forms, the description hereafter includes specific embodiments with the understanding that the disclosure is illustrative, and is not intended to limit the invention to the specific embodiments described herein.

### BRIEF DESCRIPTION OF THE FIGURES

FIG. 1 shows the concentration of tapinarof in various tissues upon administration of Compound 32 to mice, as described in Example 10.

FIG. 2 shows at left the disease activity index (DAI) of mice treated as described in Example 11, and at right the body weight change of the mice over the study days.

FIG. 3 shows the disease activity index (DAI) of mice treated as described in Example 11 at day 3 (left) and day 5 (right), with the bars indicating, from left to right, control, vehicle, Compound 31 (equivalent to 30 mpk tapinarof), tofacitinib (30 mpk), and ozanimod (1 mpk).

### DETAILED DESCRIPTION

Provided herein are compounds of Formula (I):

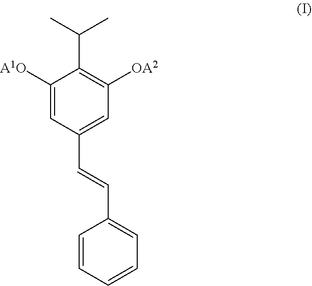

wherein A¹ and A² are as described herein, which can function as prodrugs of tapinarof. Prodrugs are compounds that are converted in the body to pharmacologically active drugs, and can be targeted to particular tissues. For example, the prodrugs described herein can release the active component, tapinarof, into targeted tissue through a variety of different enzymatic (e.g., hydrolases, esterases, peptidases, phosphatases, sulfatases) or nonenzymatic (e.g., chemical degradation with or without the aid of functional groups within the prodrug molecules) mechanisms.

Because prodrugs undergo a controlled or predictable transformation in vivo before exhibiting a therapeutic effect, prodrug formulations have become an important strategy for modulating the therapeutic effects of drugs. Employing a prodrug can enhance the therapeutic efficacy and/or reduce adverse effects of a drug via different mechanisms, including increased solubility, improved permeability and bioavailability, prolonged half-life, and tissue-targeted delivery.

Further, the properties of the prodrugs can be tuned to allow for the controlled or predictable delivery of a drug, such as tapinarof, to a tissue of interest, e.g., to the small and/or large intestine. Delivery of tapinarof to a specific tissue may allow for the targeted modulation of the AHR pathway in the tissue where tapinarof is delivered without significant concerns of systemic exposure. Accordingly, prodrug formulations of tapinarof are desirable for their enhanced therapeutic potential.

Advantageously, the prodrugs disclosed herein are suitable for oral administration. Oral administration of drugs is beneficial because it can be performed by a subject outside of a medical setting or without direct medical supervision, and can lead to improved adherence to a therapy. Although oral administration can be the simplest method of administering a drug intended to act on the gastrointestinal tract (“GI tract”), oral administration can pose challenges when delivery of a drug to the lower GI tract is desired. In particular, two main complications can arise: (i) the drug may be absorbed in the upper GI tract before arriving at the lower GI tract; (ii) the drug may be partially or fully metabolized before reaching the lower GI tract, either by diversion to the liver via the hepatic artery, or directly in the GI tract itself.

Without intending to be bound by any particular theory, the prodrugs described herein may deliver tapinarof to the lower GI tract (e.g., to the large intestine), thereby minimizing the systemic exposure of tapinarof to a subject and increasing the safety margin of a therapeutic regime. Without wishing to be bound by theory, it is believed the prodrugs described herein are stable in the stomach and small intestine and pass through the upper GI tract largely unchanged until they reach the large intestine. In the large intestine, the prodrugs can undergo a conversion to deliver tapinarof to the large intestine in a targeted manner.

The prodrugs described herein can be formulated to comprise the conjugate of tapinarof and a second therapeutic, such that when the prodrug is converted to tapinarof in e.g., the large intestine, a second drug is released. For example, a prodrug described herein can comprise the conjugate of tapinarof and 5-amino salicylic acid, which can undergo a conversion to release both tapinarof and 5-amino salicylic acid in e.g., the large intestine. Another contemplated example is a conjugate of tapinarof with a second AHR modulator, which is capable of releasing two, independent AHR chemical modulators that act via distinct mechanisms.

Also contemplated are other methods known to those skilled in the art for delaying the release and uptake of tapinarof into target tissues. Nonlimiting examples of these methods include use of an oral dosage form (e.g., tablet, capsule, or multiparticulate) having an enteric coating, which enables the release of tapinarof to be delayed until the dosage form has passed through the stomach and small intestine, as well as formulating tapinarof into a micelle, liposome, or some other encapsulated form. The preparation of such delayed release formulations can be achieved by those skilled in the art by known methods and procedures.

### Compounds of the Disclosure

In one aspect, disclosed herein are compounds having a structure of Formula (I):

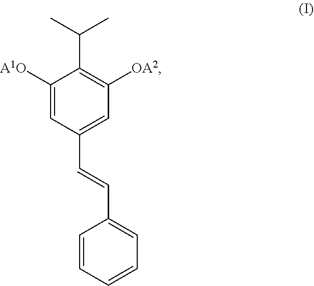

or a pharmaceutically acceptable salt thereof, wherein:
each of A¹ and A² independently is H, C₁₋₆alkyl, or

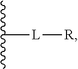

and at least one of A¹ and A² is

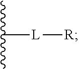

each L independently is a bond,

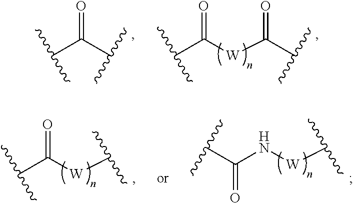

W is

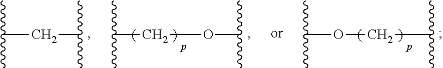

n is an integer from 1-6;
p is 2 or 3; and
each R independently is a polar group capable of forming at least two hydrogen bonds.

In some embodiments, one of A¹ and A² is H or C₁₋₆alkyl, and the other of A¹ and A² is

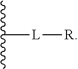

In various embodiments, one of A¹ and A² is H or CH₃, and the other of A¹ and A² is

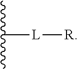

In some cases, one of A¹ and A² is H, and the other of A¹ and A² is

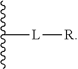

In some embodiments, one of A¹ and A² is CH₃, and the other of A¹ and A² is

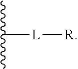

Accordingly, the compound of Formula (I) can have a structure:

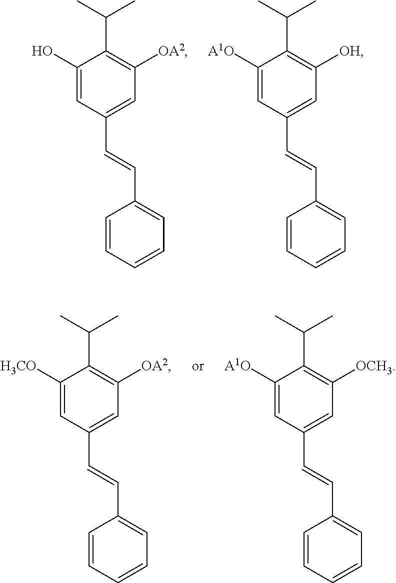

In some cases each of A¹ and A² is

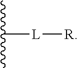

In various cases, each of A¹ and A² is

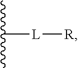

and the L groups are the same. In some embodiments, each of A¹ and A² is

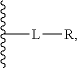

and the L groups are different. In various embodiments, each of A¹ and A² is

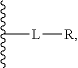

and the R groups are the same. In some embodiments, each of A¹ and A² is

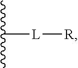

and the R groups are different. In some cases, A¹ and A² are each

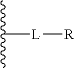

and the same. In various cases, A¹ and A² are each

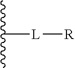

and different.

In some embodiments, one of A¹ and A² is H or C₁₋₆alkyl (e.g., H or CH₃), and the other of A¹ and A² is

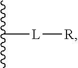

and L is a bond. In various embodiments, one of A¹ and A² is H or C₁₋₆alkyl (e.g., H or CH₃), and the other of A¹ and A² is

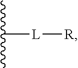

and L is a linking group (e.g.,

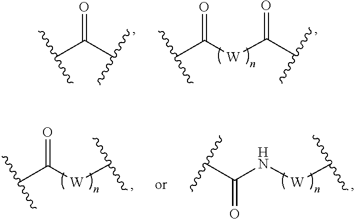

wherein W and n are as previously defined). In some cases, each of A¹ and A² is

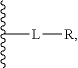

and each L is a bond. In various cases, each of A¹ and A² is

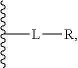

and one L is a bond and the other L is a linking group (e.g.,

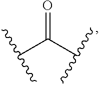

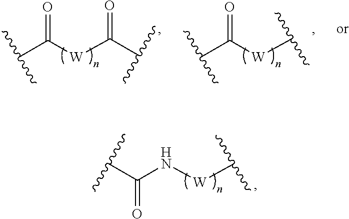

wherein W and n are as previously defined). In some embodiments, each of A¹ and A² is

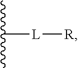

and each L is a linking group (e.g.,

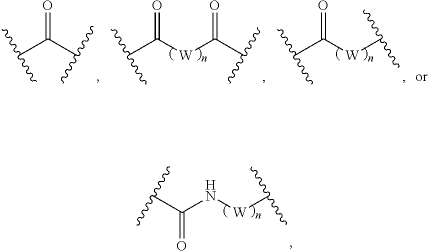

wherein W and n are as previously defined), where the linking groups can be the same or different.

The linking group L can be any group capable of connecting R (a polar group capable of forming at least two hydrogen bonds) to the oxygen atoms on the aryl ring of the compound of Formula (I) to result in an ester linkage or a carbamate linkage. Specifically contemplated linking groups (L) include

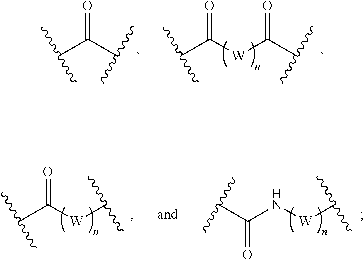

wherein W is

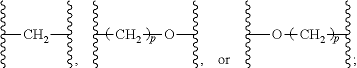

n is an integer from 1-6; and p is 2 or 3. In some cases, n is an integer from 2 to 5. In some embodiments, n is 2, 3, or 4. In various embodiments, n is 2 or 3. In some embodiments, n is 1. In various embodiments, n is 2. In some cases, n is 3. In various cases, n is 4. In some embodiments, n is 5. In various embodiments, n is 6. In some cases, W is

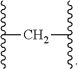 [SMILES: *C*]

In some embodiments, at least one L independently is selected from the group consisting of

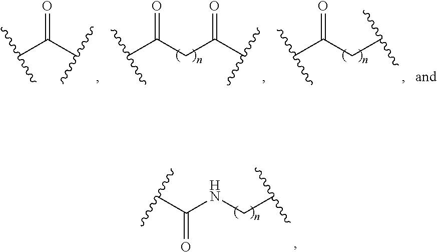

and n is 1, 2, 3, 4, 5, or 6. In some cases, at least one L independently is selected from the group consisting of

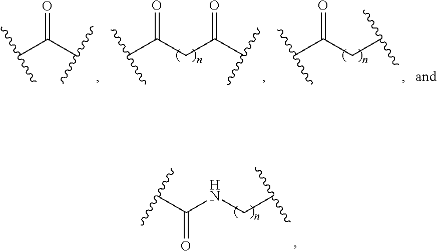

and n is 2, 3, or 4. In some cases, at least one L independently is selected from the group consisting of

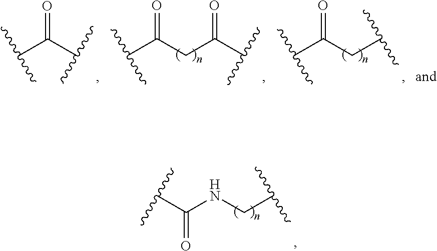

and n is 2 or 3. In some embodiments, each L is independently selected from the group consisting of

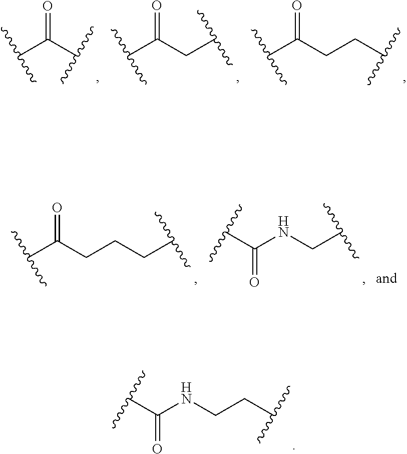

In various embodiments, W is such as

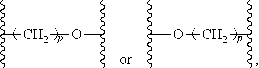

such as

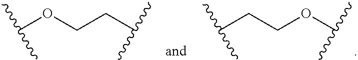 [SMILES: *CCO* ; *CCO*]

The compounds of Formula (I) comprise at least one

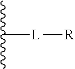

group, wherein R is a polar group capable of forming at least two hydrogen bonds. In some embodiments, R is a polar group capable of forming at least three hydrogen bonds. As used herein, a “polar group” is a group of atoms that has a net dipole moment as a result of opposing charges (e.g., a negatively charge end and a positively charged end) from polar bonds arranged asymmetrically. A “hydrogen bond” is an electrostatic force of attraction between a hydrogen bond donor and a hydrogen bond acceptor through a bridging hydrogen atom. Thus, a hydrogen bond donor and a hydrogen bond acceptor are pairs of atoms or groups of atoms that can share a hydrogen atom in a hydrogen bond. The hydrogen bond donor is an atom or group of atoms that supplies the bridging hydrogen atom of a hydrogen bond. Examples of hydrogen bond donors include, but are not limited to alcohols, amines, amides, sulfonamides, and phosphoramides, as well as unionized forms of carboxylic acids, sulfates, and phosphates, each of which comprises a hydrogen atom that is conjugated to a more electronegative atom (e.g., an oxygen or nitrogen atom) and acts as the bridging hydrogen atom of a hydrogen bond. The hydrogen bond acceptor comprises an electronegative atom with a lone pair of electrons (e.g., O or N) that can form a hydrogen bond with the bridging hydrogen atom supplied by the corresponding hydrogen bond donor. Examples of hydrogen bond acceptors include, but are not limited to alcohols, amines, ethers, carbonyls, carboxylic acids, sulfates, sulfonamides, phosphates, and phosphoramides, each of which can supply a lone pair of electrons to a hydrogen atom of a corresponding hydrogen bond donor. Accordingly, as used herein, a “polar group capable of forming at least two hydrogen bonds” is a group of atoms that has a net dipole moment as a result of opposing charges and a combination of at least two hydrogen bond donors and hydrogen bond acceptors.

In some embodiments, R can comprise one or more of an alcohol, amine, amide, thiol, ether, sulfide, carbonyl, sulfoxide, sulfone, carboxylic acid, sulfate, phosphate, sulfonamide, and phosphoramide, provided that R includes a combination of at least two hydrogen bond donors and acceptors. In some cases, R is selected from the group consisting of:

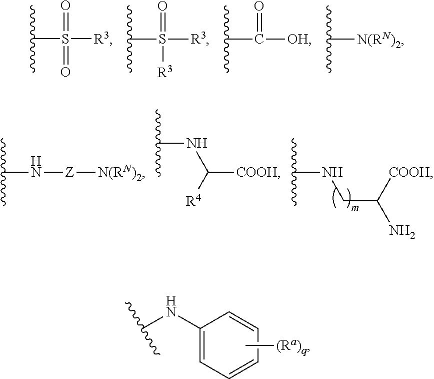

and a saccharide; wherein:
m is 1, 2, 3, 4, 5, or 6;
each R³ independently is OH or NH₂;
R⁴ is H, C₁₋₆alkyl, or

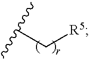

wherein: r is an integer from 1-6 and R⁵ is selected from the group consisting of

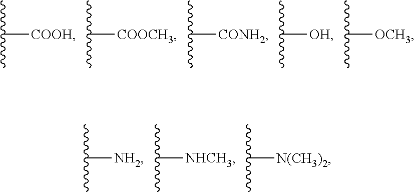

heteroaryl comprising 5 or 6 total ring atoms and 1, 2, or 3 heteroatoms selected from N, O, and S (e.g.,

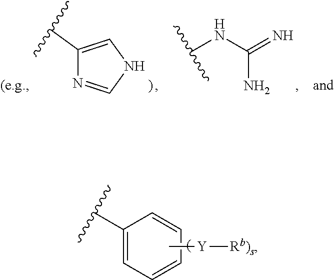

wherein Y is absent or C₁₋₂alkylene; s is 1, 2, 3, 4, or 5; and R^{b} is selected from the group consisting of

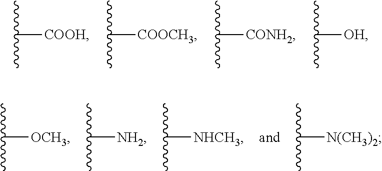

each R^{a} independently is

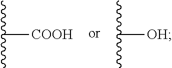

and q is 2, 3, 4, or 5;
each R^{N} independently is H or CH₃; and
Z is C₂₋₆alkylene or C₂₋₆polyoxyalkene.

In some embodiments, at least one L is a bond and R is

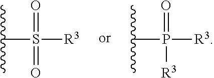

In some cases, each L is a bond and each R independently is

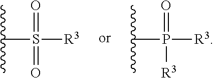

In various cases, each R independently is selected from the group consisting of

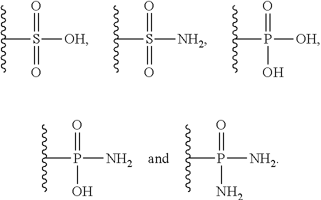

In various embodiments, each R independently is

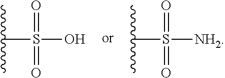 [SMILES: *S(=O)(=O)O ; "or" ; *S(N)(=O)=O]

In some cases, each R independently is

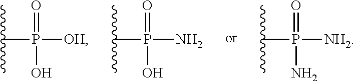

In some embodiments, one of A¹ and A² is H or C₁₋₆alkyl (e.g., H or CH₃), and the other of A¹ and A² is

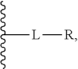

wherein L is a bond, and R is

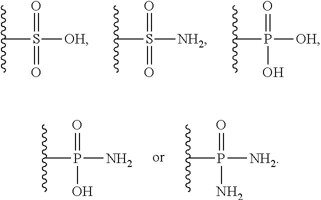

In some embodiments, one of A¹ and A² is H or C₁₋₆alkyl (e.g., H or CH₃), and the other of A¹ and A² is

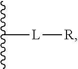

wherein L is a bond, and R is

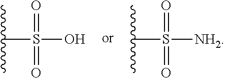 [SMILES: *S(=O)(=O)O ; "or" ; *S(N)(=O)=O]

In some embodiments, one of A¹ and A² is H or C₁₋₆alkyl (e.g., H or CH₃), and the other of A¹ and A² is

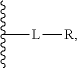

wherein L is a bond, and R is

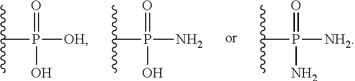

In some cases, one of A¹ and A² is H or C₁₋₆alkyl (e.g., H or CH₃), and the other of A¹ and A² is

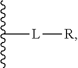

wherein L is a bond, and R is

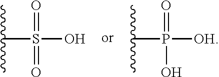 [SMILES: *S(=O)(=O)O ; "or" ; *P(=O)(O)O]

In various embodiments, each of A¹ and A² independently is

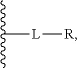

and at least one L is a bond. In some embodiments, each of A¹ and A² independently is

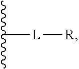

and each L is a bond. When L is a bond, each R independently can be selected from the group consisting of

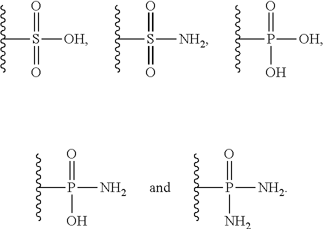 [SMILES: *S(=O)(=O)O ; *S(N)(=O)=O ; *P(=O)(O)O ; *P(N)(=O)O ; *P(N)(N)=O]

In some embodiments, each of A¹ and A² is selected from the group consisting of

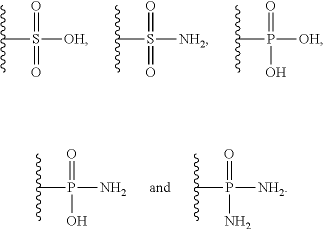 [SMILES: *S(=O)(=O)O ; *S(N)(=O)=O ; *P(=O)(O)O ; *P(N)(=O)O ; *P(N)(N)=O]

In various cases, each of A¹ and A² independently is

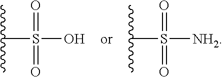 [SMILES: *S(=O)(=O)O ; *S(N)(=O)=O]

In some embodiments, each of A¹ and A² is

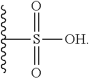 [SMILES: *S(=O)(=O)O]

In various embodiments, each of A¹ and A² is

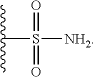 [SMILES: *S(N)(=O)=O]

In some cases, each of A¹ and A² independently is

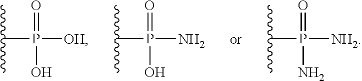 [SMILES: *P(=O)(O)O ; *P(N)(=O)O ; *P(N)(N)=O]

In various cases, each of A¹ and A² is

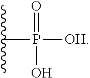 [SMILES: *P(=O)(O)O]

In some embodiments, each of A¹ and A² is

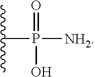 [SMILES: *P(N)(=O)O]

In various embodiments, each of A¹ and A² is

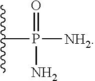 [SMILES: *P(N)(N)=O]

In some embodiments, at least one L is

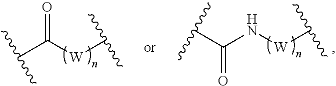

wherein W and n are as previously described, and at least one R is

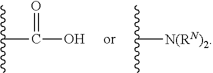

In various embodiments, each L is

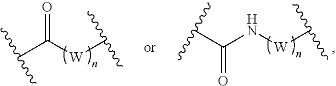

and each R is

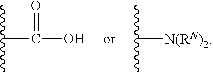

In some cases at least one R^{N} is CH₃. In various cases, each R^{N} is CH₃. In some embodiments at least one R^{N} is H. In various cases, each R^{N} is H. In some cases, at least one R is

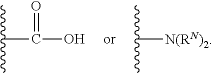

In various cases, each R independently is

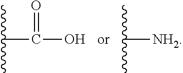

In some embodiments, n is 2, 3, or 4. In some cases, n is 2. In various cases, n is 3. In some embodiments, n is 4. In various embodiments, W is

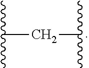 [SMILES: *C*]

In various cases, W is

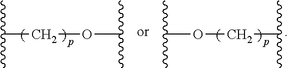

In some embodiments p is 2. In various embodiments, p is 3. In some cases, W is

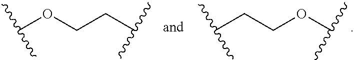 [SMILES: *CCO* ; *CCO*]

In some cases, W is selected from the group consisting of

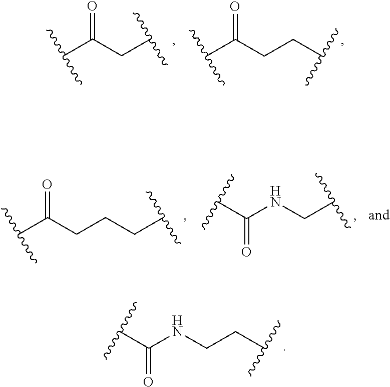 [SMILES: *CC(*)=O ; *CCC(*)=O ; *CCCC(*)=O ; *CNC(*)=O ; *CCNC(*)=O]

In some embodiments, one or both of A¹ and A² independently is selected from the group consisting of

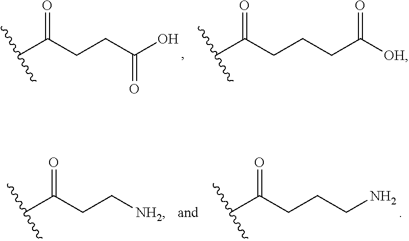 [SMILES: *C(=O)CCC(=O)O ; *C(=O)CCCC(=O)O ; *C(=O)CCN ; *C(=O)CCCN]

In some cases, one of A¹ and A² is H or C₁₋₆alkyl (e.g., H or CH₃), and the other of A¹ and A² is selected from the group consisting of

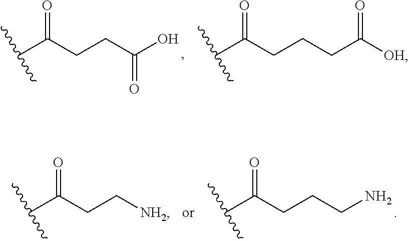 [SMILES: *C(=O)CCC(=O)O ; *C(=O)CCCC(=O)O ; *C(=O)CCN ; *C(=O)CCCN]

In various cases, at least one of A¹ and A² is

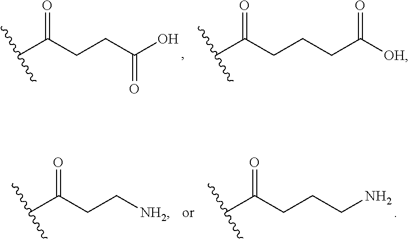 [SMILES: *C(=O)CCC(=O)O ; *C(=O)CCCC(=O)O ; *C(=O)CCN ; *C(=O)CCCN]

In some cases, each of A¹ and A² is

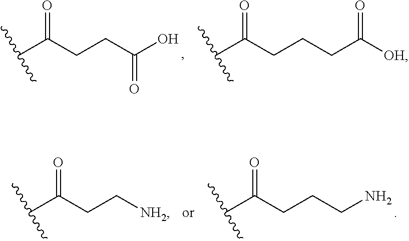 [SMILES: *C(=O)CCC(=O)O ; *C(=O)CCCC(=O)O ; *C(=O)CCN ; *C(=O)CCCN]

In some cases, at least one L is

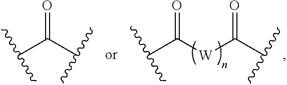

wherein W and n are as previously described, and at least one R is

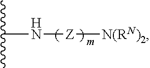

a natural or unnatural amino acid or derivative thereof (e.g.,

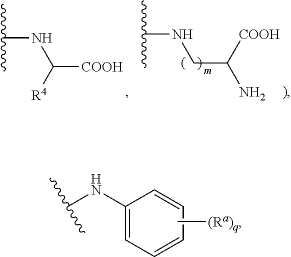

or a saccharide. In some cases, W is

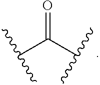 [SMILES: *C(*)=O]

In various cases, n is 2, 3, or 4. In some cases, n is 2. In various embodiments, n is 3. In some embodiments, n is 4. In various embodiments, W is

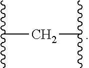 [SMILES: *C*]

In various cases, W is

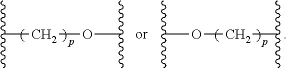

In some embodiments p is 2. In various embodiments, p is 3. In some cases, W is

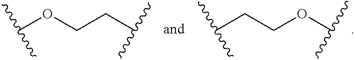 [SMILES: *CCO* ; *CCO*]

In some cases, W is selected from the group consisting of

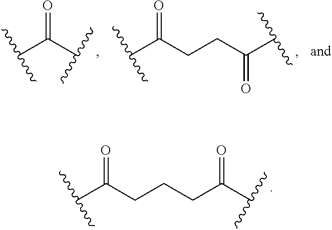

In some embodiments, at least one R is

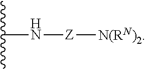

In some cases at least one R^{N} is CH₃. In various cases, each R^{N} Is CH₃. In some embodiments at least one R^{N} is H. In various cases, each R^{N} is H. In various embodiments, Z is C₂₋₆alkylene. Non-limiting examples of C₂₋₆alkylene is a straight-chain alkylene group, such as ethylene, propylene, butylene, pentylene, and hexylene. In various embodiments, C₂₋₆alkylene is a branched alkylene group, such as 2-methylethylene, 2-methylpropylene, and 2-methyl-butylene. In some embodiments, Z is ethylene or propylene. In various embodiments, Z is 2-methylethylene or 2-methylpropylene. In various cases Z is C₂₋₆polyoxyalkylene. Non-limiting examples of C₂₋₆polyoxyalkylene include a polyethylene glycol moiety and a polypropylene glycol moiety, such as C₂polyoxyalkylene (e.g., —CH₂CH₂OCH₂CH₂O— and —CH₂CH₂CH₂OCH₂CH₂CH₂O—). In some embodiments, R can be

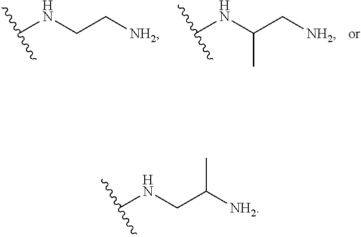

In some cases, one of A¹ and A² is H or C₁₋₆alkyl (e.g., H or CH₃), and the other of A¹ and A² is selected from the group consisting of

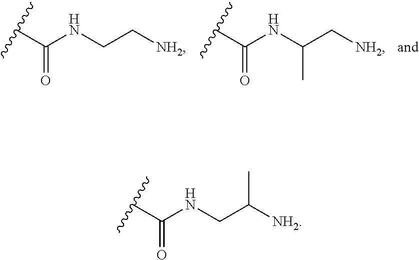

In various cases, at least one of A¹ and A² is

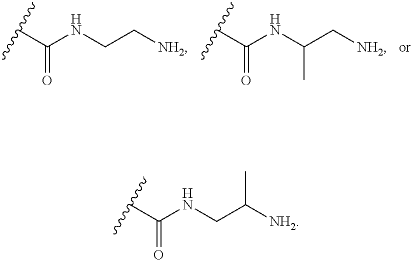

In some cases, each of A¹ and A² is

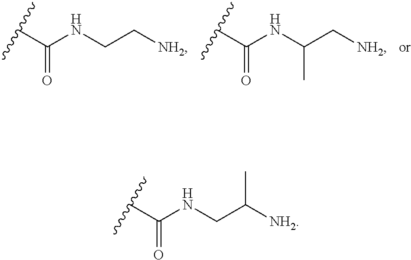

In various cases, at least one R is an amino acid (e.g., one of the 20 natural amino acids) or a derivative thereof (e.g., an unnatural amino acid or an analog of a natural amino acid). Examples of amino acid derivatives include, but are not limited to β-amino acids, D-amino acids, homo-amino acids, β-homo-amino acids, N-methyl amino acids, and α-methyl amino acids, such as ornithine. Thus, in some embodiments, R can be

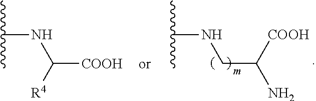

In various embodiments, m is 1-4. In some cases, m is 1. In various cases, m is 2. In some embodiments, m is 3. In various embodiments, m is 4. In various cases, R is

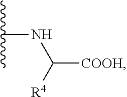

wherein R⁴ is H, C₁₋₆alkyl, or

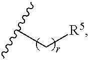

and r and R⁵ are as previously described herein. In some embodiments, R⁴ is H. In various embodiments, R⁴ is C₁₋₆alkyl. For example, R⁴ can be methyl, ethyl, propyl, isopropyl, 1-methylpropyl, 2-methylpropyl. In some embodiments, R⁴ is methyl. In some cases, R⁴ is

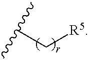

In some embodiments, r is an integer from 1-4. In various embodiments, r is 1. In some cases, r is 2. In various embodiments, r is 3. In some cases, r is 4. In some embodiments, R⁵ can be selected from the group consisting of from the group consisting of

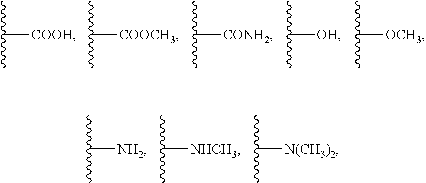

heteroaryl comprising 5 or 6 total ring atoms and 1, 2, or 3 heteroatoms selected from N, O, and S (e.g.,

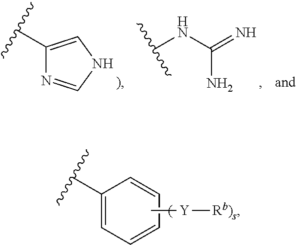

wherein Y is absent or C₁₋₂alkylene; s is 1, 2, 3, 4, or 5; and R^{b} is selected from the group consisting of

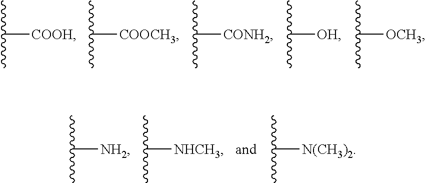

In some embodiments, R⁴ is

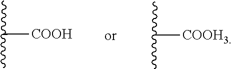

In some embodiments, R⁴ is

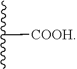 [SMILES: *C(=O)O]

In some cases, R⁴ is

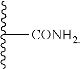 [SMILES: *C(N)=O]

In some cases, R⁴ is

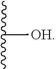 [SMILES: *O]

In various cases, R⁴ is

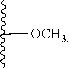 [SMILES: *OC]

In some embodiments, R⁴ is

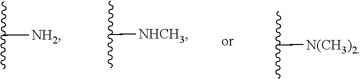

In some cases, R⁴ is

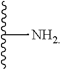 [SMILES: *N]

In various embodiments, R⁴ is

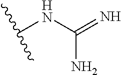 [SMILES: *NC(=N)N]

or heteroaryl comprising 5 or 6 total ring atoms and 1, 2, or 3 heteroatoms selected from N, O, and S. In some embodiments, R⁴ is

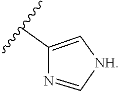 [SMILES: *c1c[nH]cn1]

In various cases, R⁴ is

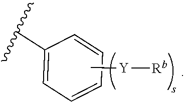

In some cases, Y is absent. In various cases, Y is

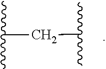 [SMILES: *C*]

In various cases, Y is

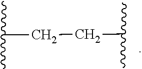 [SMILES: *CC*]

In some embodiments, s is 1 or 2 and each R^{b} independently is selected from the group consisting of

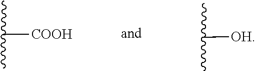

In some cases, s is 1 or 2 and R⁴ is

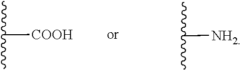

In some embodiments R⁴ is

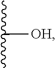 [SMILES: *O]

In various embodiments, each R independently is selected from the group consisting of

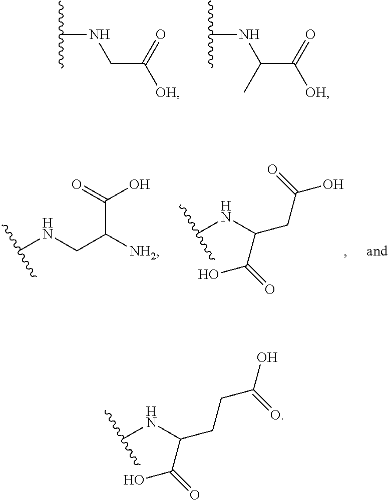

In some cases, one of A¹ and A² is H or C₁₋₆alkyl (e.g., H or CH₃), and the other of A¹ and A² is

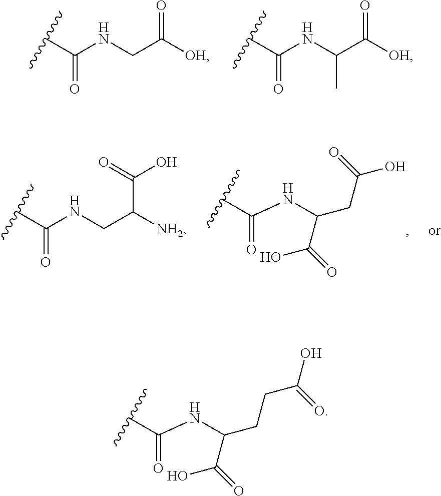

In various cases, at least one of A¹ and A² is

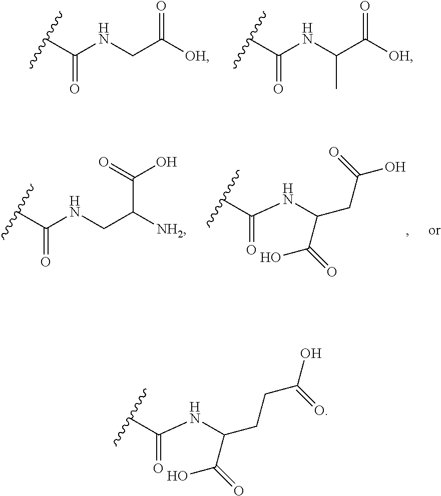

In some cases, each of A¹ and A² is

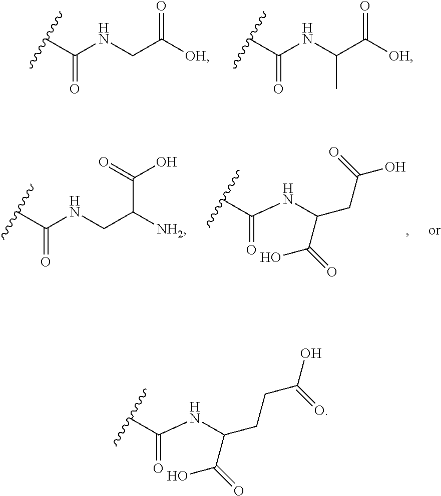

In some cases, R can be

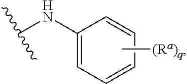

wherein q is 2, 3, 4, or 5; and each R^{a} independently is

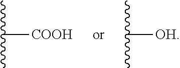

In some embodiments, q is 2 or 3. In various cases, q is 2. In some embodiments, a is 3. In various embodiments, q is 4. In some cases, q is 5. In various cases, one of A¹ and A² is H or C₁₋₆alkyl (e.g., H or CH₃), and the other of A¹ and A² is

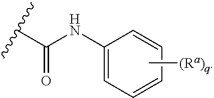

In some cases, at least one of A¹ and A² is

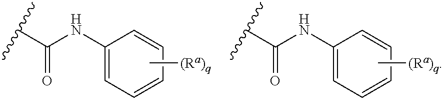

In some cases, each of A¹ and A² is

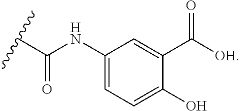 [SMILES: *C(=O)Nc1ccc(O)c(C(=O)O)c1]

In some cases, R can be a saccharide. The saccharide can be any saccharide known in the art, such as a monosaccharide (e.g., a simple sugar having a general formula C₆H₁₂O₆), a disaccharide (e.g., a saccharide comprising two monosaccharides linked together by a glycosidic bond), an oligosaccharide (e.g., a saccharide comprising 3-10 monosaccharides linked together by glycosidic bonds), a polysaccharide (e.g., a saccharide comprising seven or more monosaccharides linked together by glycosidic bonds), or an amino saccharide (e.g., a saccharide in which a hydroxyl group has been replace with an amino group). As used herein, a glycosidic bond is bond between a hemiacetal or hemiketal group of a saccharide (or a molecule derived from a saccharide) and the hydroxyl group of a second saccharide. Each glycosidic bond can independently be an α-glycosidic bond (e.g., the carbon atoms of each partner of the glycosidic bond have the same stereochemistry) or a β-glycosidic bond (e.g., the carbon atoms of each partner of the glycosidic bond have different stereochemistry). In some cases, the oligosaccharide comprises α-glycosidic bonds. In some embodiments, the saccharide is a monosaccharide, a disaccharide, an oligosaccharide, or an aminosaccharide. Contemplated monosaccharides include, but are not limited to, glucose, fructose, galactose, ribose, and xylose. In various embodiments, the saccharide is a disaccharide. Contemplated disaccharides include, but are not limited to, sucrose, lactose, lactulose, trehalose, and maltose. In some cases, the saccharide is an oligosaccharide comprising 3-10 monosaccharide units. In various cases, the oligosaccharide is a straight-chain oligosaccharide. In some embodiments, the oligosaccharide is a cyclic oligosaccharide. In some cases, the saccharide (e.g., the oligosaccharide) comprises pyranose units. In various cases, the saccharide is a cyclic oligosaccharide comprising 5-8 pyranose units. In some cases, the pyranose units comprise glucose. In some embodiments, the oligosaccharide comprises α-1,4 glycosidic bonds. In some embodiments, the oligosaccharide is a cyclodextrin. In some cases, R is

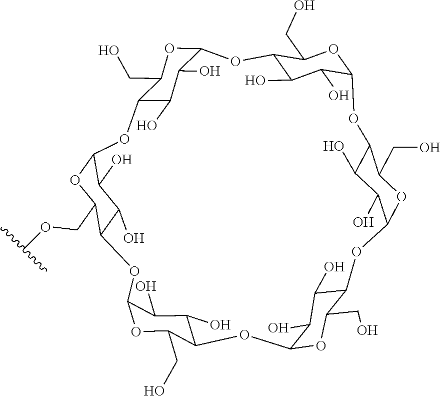 [SMILES: *OCC1OC2OC3C(CO)OC(OC4C(CO)OC(OC5C(CO)OC(OC6C(CO)OC(OC7C(CO)OC(OC1C(O)C2O)C(O)C7O)C(O)C6O)C(O)C5O)C(O)C4O)C(O)C3O]

In various cases, R has a stereochemical configuration as shown:

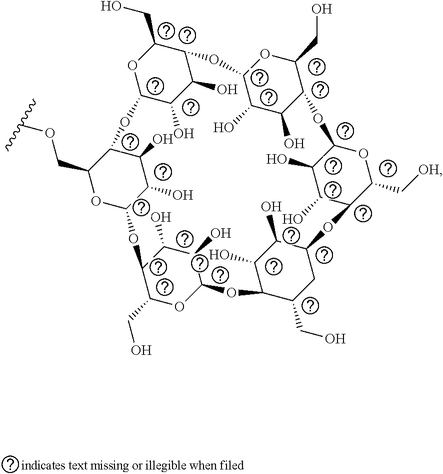 [SMILES: *OC[C@@H]1O[C@H]2O[C@H]3[C@H](O)[C@@H](O)[C@@H](O[C@@H]4[C@@H](CO)C[C@H](O[C@H]5[C@H](O)[C@@H](O)[C@@H](O[C@@H]6[C@@H](O)[C@H](O)[C@H](O[C@@H]7[C@@H](O)[C@H](O)[C@H](O[C@@H]1[C@@H](O)[C@@H]2O)O[C@H]7CO)O[C@H]6CO)O[C@@H]5CO)[C@H](O)[C@H]4O)O[C@@H]3CO]

In some cases, one of A¹ and A² is H or C₁₋₆alkyl (e.g., H or CH₃), and the other of A¹ and A² is

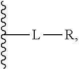

wherein L

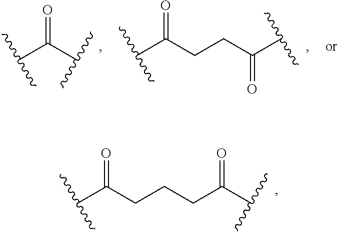

and R is a saccharide. In some cases, one or both of A¹ and A² is

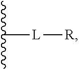

wherein each L independently is

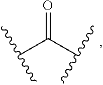 [SMILES: *C(*)=O]

and R is a saccharide. In some cases, one or both of A¹ and A² (e.g., at least one of A¹ and A² or each of A¹ and A²) is

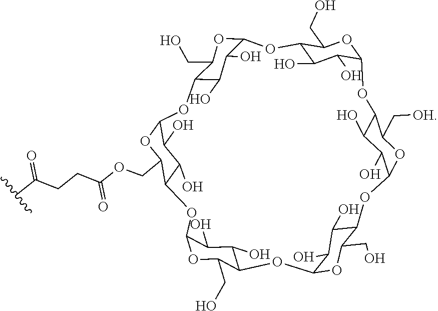 [SMILES: *C(=O)CCC(=O)OCC1OC2OC3C(CO)OC(OC4C(CO)OC(OC5C(CO)OC(OC6C(CO)OC(OC7C(CO)OC(OC1C(O)C2O)C(O)C7O)C(O)C6O)C(O)C5O)C(O)C4O)C(O)C3O]

Contemplated compounds of Formula (I) include, but are not limited to the compounds listed in Table 1:

TABLE 1

| Compound | Structure |
|---|---|
| 1 | 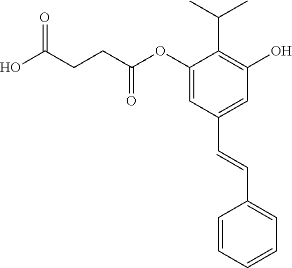 [SMILES: CC(C)c1c(O)cc(/C=C/c2ccccc2)cc1OC(=O)CCC(=O)O] |
| 2 | 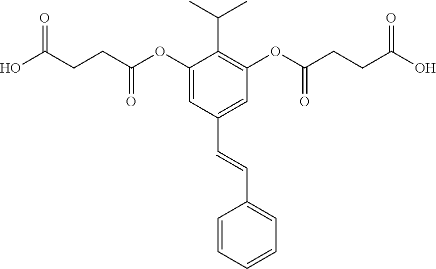 [SMILES: CC(C)c1c(OC(=O)CCC(=O)O)cc(/C=C/c2ccccc2)cc1OC(=O)CCC(=O)O] |
| 3 | 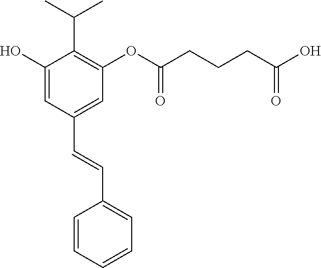 [SMILES: CC(C)c1c(O)cc(/C=C/c2ccccc2)cc1OC(=O)CCCC(=O)O] |
| 4 | 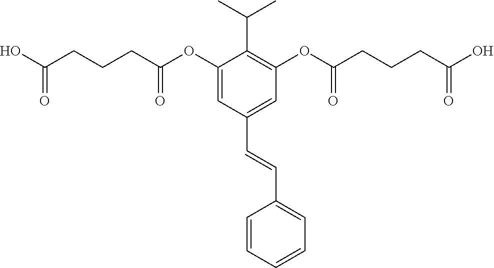 [SMILES: CC(C)c1c(OC(=O)CCCC(=O)O)cc(/C=C/c2ccccc2)cc1OC(=O)CCCC(=O)O] |
| 5 | 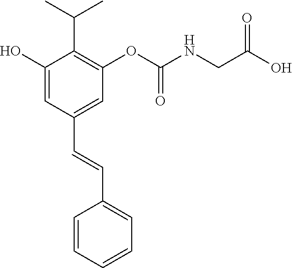 [SMILES: CC(C)c1c(O)cc(/C=C/c2ccccc2)cc1OC(=O)NCC(=O)O] |
| 6 | 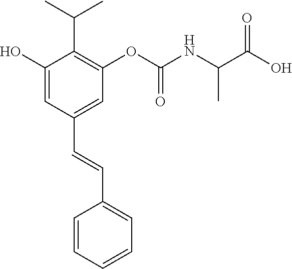 [SMILES: CC(NC(=O)Oc1cc(/C=C/c2ccccc2)cc(O)c1C(C)C)C(=O)O] |
| 7 | 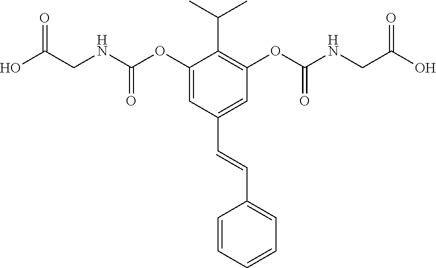 [SMILES: CC(C)c1c(OC(=O)NCC(=O)O)cc(/C=C/c2ccccc2)cc1OC(=O)NCC(=O)O] |
| 8 | 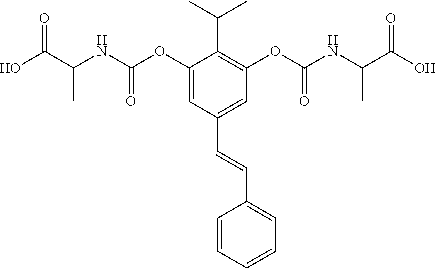 [SMILES: CC(NC(=O)Oc1cc(/C=C/c2ccccc2)cc(OC(=O)NC(C)C(=O)O)c1C(C)C)C(=O)O] |
| 9 | 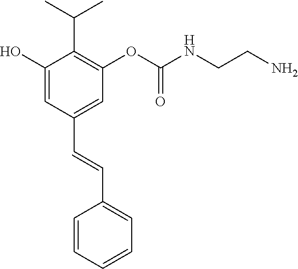 [SMILES: CC(C)c1c(O)cc(/C=C/c2ccccc2)cc1OC(=O)NCCN] |
| 10 | 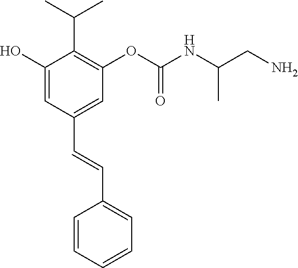 [SMILES: CC(CN)NC(=O)Oc1cc(/C=C/c2ccccc2)cc(O)c1C(C)C] |
| 11 | 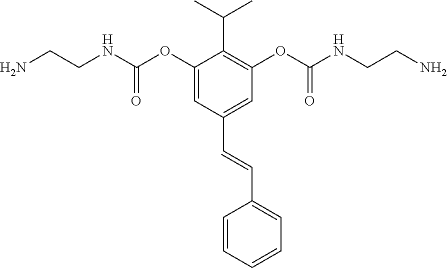 [SMILES: CC(C)c1c(OC(=O)NCCN)cc(/C=C/c2ccccc2)cc1OC(=O)NCCN] |
| 12 | 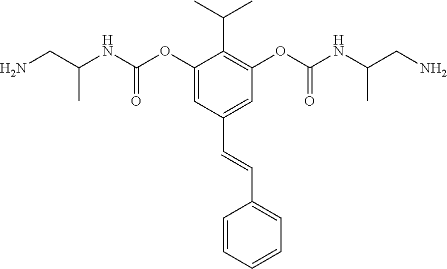 [SMILES: CC(CN)NC(=O)Oc1cc(/C=C/c2ccccc2)cc(OC(=O)NC(C)CN)c1C(C)C] |
| 13 | 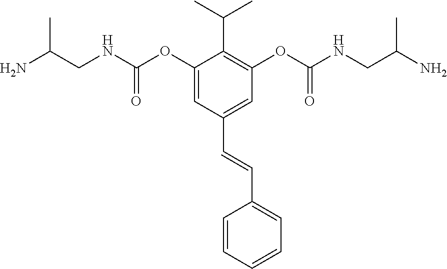 [SMILES: CC(N)CNC(=O)Oc1cc(/C=C/c2ccccc2)cc(OC(=O)NCC(C)N)c1C(C)C] |
| 14 | 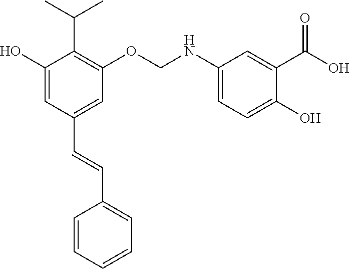 [SMILES: CC(C)c1c(O)cc(/C=C/c2ccccc2)cc1OCNc1ccc(O)c(C(=O)O)c1] |
| 15 | 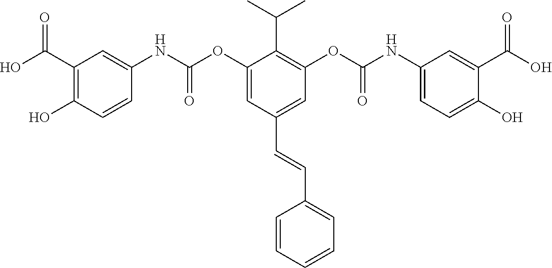 [SMILES: CC(C)c1c(OC(=O)Nc2ccc(O)c(C(=O)O)c2)cc(/C=C/c2ccccc2)cc1OC(=O)Nc1ccc(O)c(C(=O)O)c1] |
| 16 | 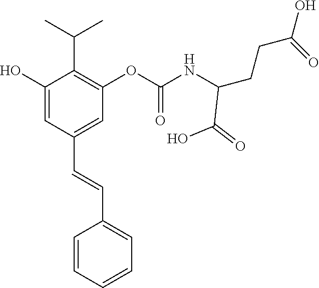 [SMILES: CC(C)c1c(O)cc(/C=C/c2ccccc2)cc1OC(=O)NC(CCC(=O)O)C(=O)O] |
| 17 | 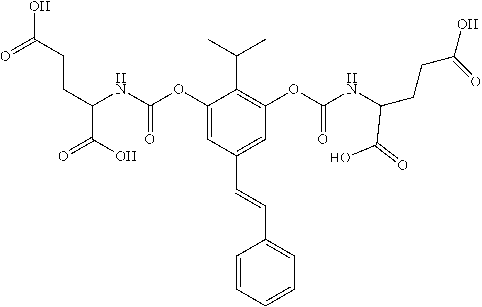 [SMILES: CC(C)c1c(OC(=O)NC(CCC(=O)O)C(=O)O)cc(/C=C/c2ccccc2)cc1OC(=O)NC(CCC(=O)O)C(=O)O] |
| 18 | 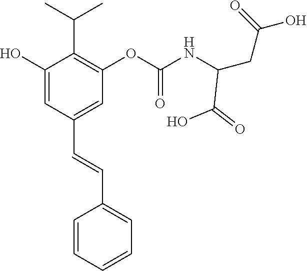 [SMILES: CC(C)c1c(O)cc(/C=C/c2ccccc2)cc1OC(=O)NC(CC(=O)O)C(=O)O] |
| 19 | 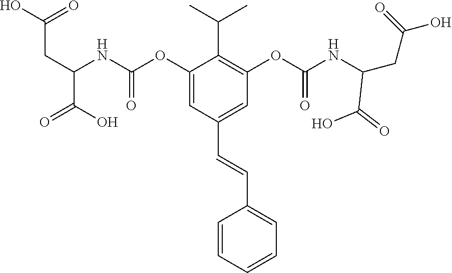 [SMILES: CC(C)c1c(OC(=O)NC(CC(=O)O)C(=O)O)cc(/C=C/c2ccccc2)cc1OC(=O)NC(CC(=O)O)C(=O)O] |
| 20 | 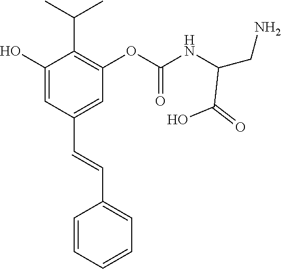 [SMILES: CC(C)c1c(O)cc(/C=C/c2ccccc2)cc1OC(=O)NC(CN)C(=O)O] |
| 21 | 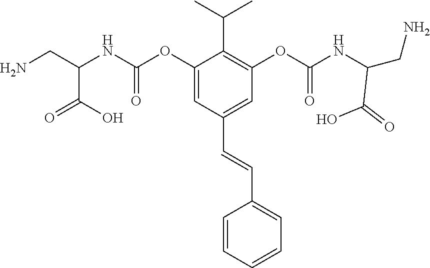 [SMILES: CC(C)c1c(OC(=O)NC(CN)C(=O)O)cc(/C=C/c2ccccc2)cc1OC(=O)NC(CN)C(=O)O] |
| 22 | 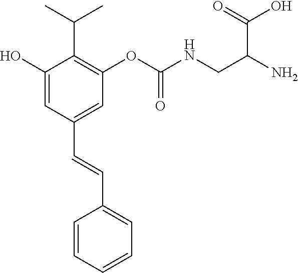 [SMILES: CC(C)c1c(O)cc(/C=C/c2ccccc2)cc1OC(=O)NCC(N)C(=O)O] |
| 23 | 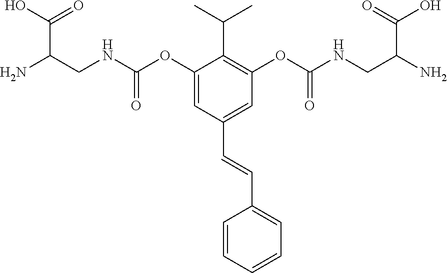 [SMILES: CC(C)c1c(OC(=O)NCC(N)C(=O)O)cc(/C=C/c2ccccc2)cc1OC(=O)NCC(N)C(=O)O] |
| 24 | 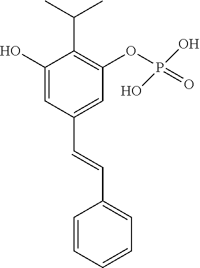 [SMILES: CC(C)c1c(O)cc(/C=C/c2ccccc2)cc1OP(=O)(O)O] |
| 25 | 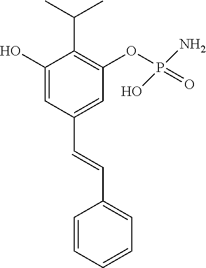 [SMILES: CC(C)c1c(O)cc(/C=C/c2ccccc2)cc1OP(N)(=O)O] |
| 26 | 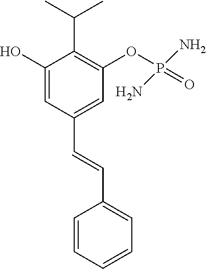 [SMILES: CC(C)c1c(O)cc(/C=C/c2ccccc2)cc1OP(N)(N)=O] |
| 27 | 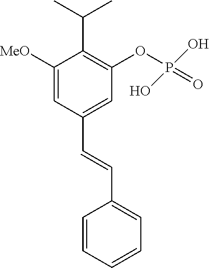 [SMILES: COc1cc(/C=C/c2ccccc2)cc(OP(=O)(O)O)c1C(C)C] |
| 28 | 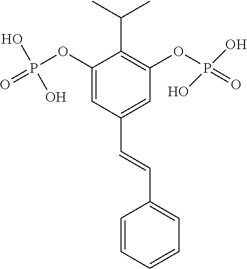 [SMILES: CC(C)c1c(OP(=O)(O)O)cc(/C=C/c2ccccc2)cc1OP(=O)(O)O] |
| 29 | 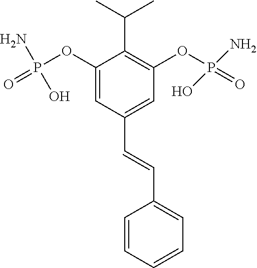 [SMILES: CC(C)c1c(OP(N)(=O)O)cc(/C=C/c2ccccc2)cc1OP(N)(=O)O] |
| 30 | 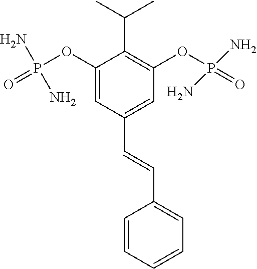 [SMILES: CC(C)c1c(OP(N)(N)=O)cc(/C=C/c2ccccc2)cc1OP(N)(N)=O] |
| 31 | 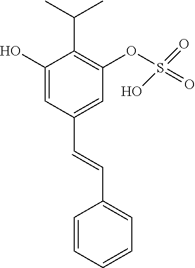 [SMILES: CC(C)c1c(O)cc(/C=C/c2ccccc2)cc1OS(=O)(=O)O] |
| 32 | 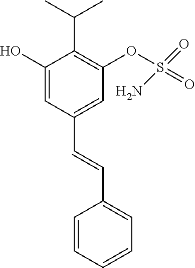 [SMILES: CC(C)c1c(O)cc(/C=C/c2ccccc2)cc1OS(N)(=O)=O] |
| 33 | 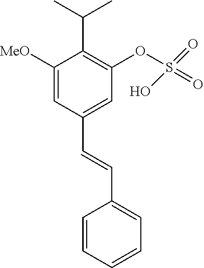 [SMILES: COc1cc(/C=C/c2ccccc2)cc(OS(=O)(=O)O)c1C(C)C] |
| 34 | 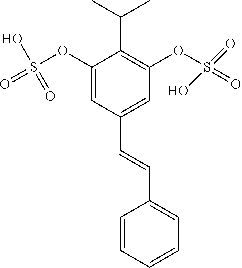 [SMILES: CC(C)c1c(OS(=O)(=O)O)cc(/C=C/c2ccccc2)cc1OS(=O)(=O)O] |
| 35 | 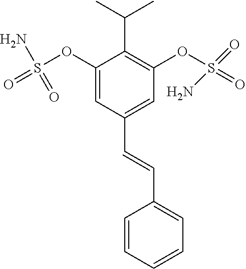 [SMILES: CC(C)c1c(OS(N)(=O)=O)cc(/C=C/c2ccccc2)cc1OS(N)(=O)=O] |
| 36 | 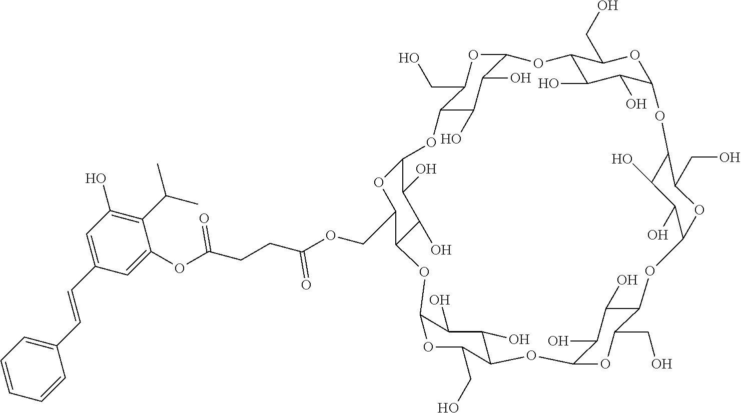 [SMILES: CC(C)c1c(O)cc(/C=C/c2ccccc2)cc1OC(=O)CCC(=O)OCC1OC2OC3C(CO)OC(OC4C(CO)OC(OC5C(CO)OC(OC6C(CO)OC(OC7C(CO)OC(OC1C(O)C2O)C(O)C7O)C(O)C6O)C(O)C5O)C(O)C4O)C(O)C3O] |

In some embodiments, the compounds of Formula (I) are selected from the group consisting of compounds 1-4, or pharmaceutically acceptable salts thereof. In various embodiments, the compounds of Formula (I) are selected from the group consisting of compounds 5-8, or pharmaceutically acceptable salts thereof. In some cases, the compounds of Formula (I) are selected from the group consisting of compounds 9-13 and 16-23, or pharmaceutically acceptable salts thereof. In various cases, the compounds of Formula (I) are compound 14 or compound 15, or pharmaceutically acceptable salts thereof. In some embodiments, the compounds of Formula (I) are selected from the group consisting of compounds 24-35, or pharmaceutically acceptable salts thereof. In various embodiments, the compounds of Formula (I) is compound 36, or a pharmaceutically acceptable salt thereof.

Unless otherwise indicated, structures depicted herein are also meant to include all isomeric (e.g., enantiomeric, diastereomeric, cis-trans, conformational, and rotational) forms of the structure. For example, the R and S configurations for each asymmetric center, (Z) and (E) double bond isomers, and (Z) and (E) conformational isomers are included in this disclosure, unless only one of the isomers is specifically indicated. Therefore, single stereochemical isomers as well as enantiomeric, diastereomeric, cis/trans, conformational, and rotational mixtures of the present compounds are within the scope of the disclosure. In some cases, the compounds disclosed herein are stereoisomers. “Stereoisomers” refer to compounds that differ in the chirality of one or more stereocenters. Stereoisomers include enantiomers and diastereomers. The compounds disclosed herein can exist as a single stereoisomer, or as a mixture of stereoisomers. Stereochemistry of the compounds shown herein indicate a relative stereochemistry, not absolute, unless discussed otherwise. As indicated herein, a single stereoisomer, diastereomer, or enantiomer refers to a compound that is at least more than 50% of the indicated stereoisomer, diastereomer, or enantiomer, and in some cases, at least 90% or 95% of the indicated stereoisomer, diastereomer, or enantiomer.

Thus, the compounds of Formula (I) can exhibit E or Z (not shown) stereochemistry at the double bond. in some embodiments, the compounds of Formula (I) exhibit E stereochemistry at the double bond. In various embodiments, the compounds of Formula (I) exhibit Z stereochemistry at the double bond. The compounds of Formula (I) can have any stereochemical configuration at the sp³ carbon atoms. In some embodiments, the compounds of the disclosure are optically pure. As used herein, “optically pure” refers to the presence of only one enantiomer of a compound if multiple stereochemical configurations can exist. In various embodiments, the chiral moieties present in the compounds of the disclosure are derived from either natural or unnatural amino acids or saccharides. In some embodiments, the compounds of Formula (I) have a stereochemical configuration as follows:

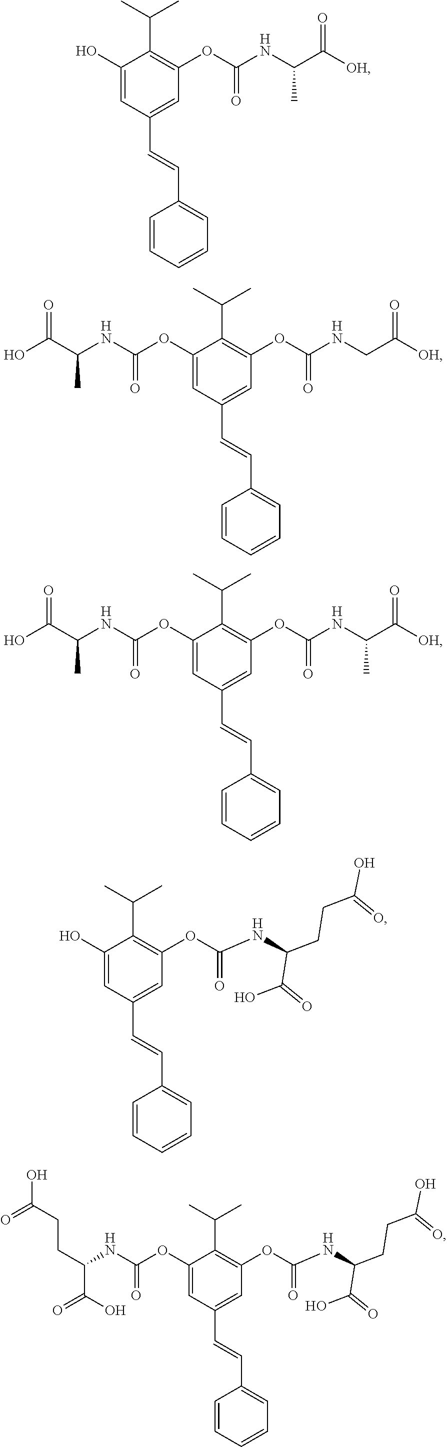

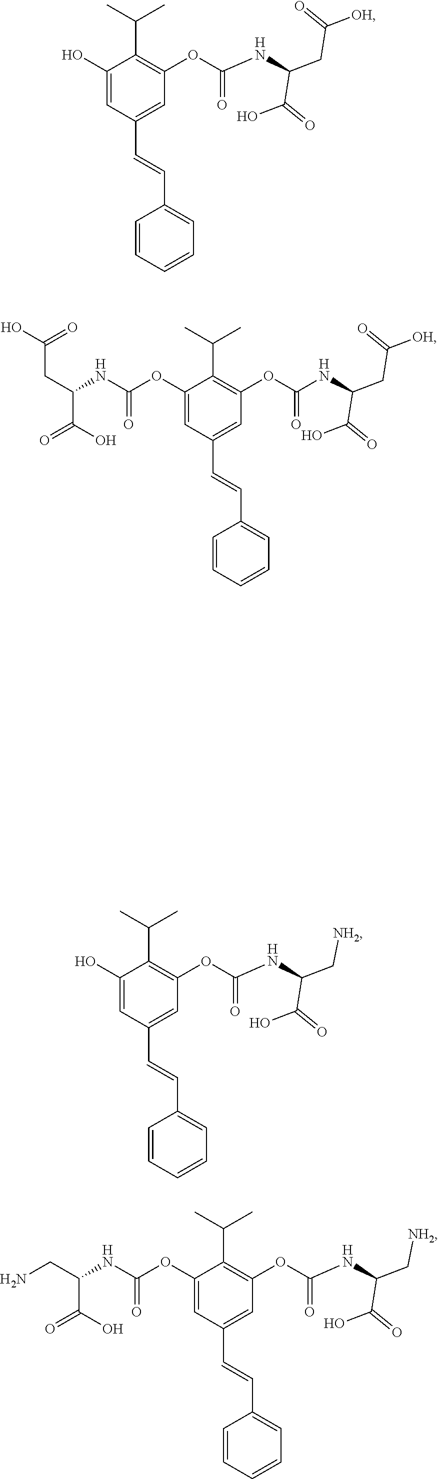

or a pharmaceutically acceptable salt thereof.

Unless otherwise indicated, all tautomeric forms of the compounds of the disclosure are within the scope of the disclosure.

Additionally, unless otherwise indicated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C-or ¹⁴C-enriched carbon are within the scope of this disclosure. Such compounds are useful, for example, as analytical tools or probes in biological assays. Such compounds, especially deuterium analogs, can also be therapeutically useful. Thus, further disclosed herein are deuterated compounds or salts of Formula (I), in which one or more isotopes of hydrogen have been replaced with deuterium.

The compounds of the disclosure are defined herein by their chemical structures and/or chemical names. Where a compound is referred to by both a chemical structure and a chemical name, and the chemical structure and chemical name conflict, the chemical structure is determinative of the compound's identity.

As used herein, “alkyl” refers to straight chained and branched saturated hydrocarbon groups containing one to thirty carbon atoms, for example, one to four carbon atoms (e.g., 1, 2, 3, or 4). The term Cₙ means the alkyl group has “n” carbon atoms. For example, C₃ alkyl refers to an alkyl group that has 3 carbon atoms. C₁₋₄alkyl refers to an alkyl group having a number of carbon atoms encompassing the entire range (i.e., 1 to 4 carbon atoms), as well as all subgroups (e.g., 1-2, 1-3, 2-3, 2-4, 1, 2, 3, and 4 carbon atoms). Nonlimiting examples of alkyl groups include, methyl, ethyl, n-propyl, isopropyl, n-butyl, sec-butyl (2-methylpropyl), and t-butyl (1,1-dimethylethyl). Unless otherwise indicated, an alkyl group can be an unsubstituted alkyl group or a substituted alkyl group.

As used herein, “alkylene” refers to a bivalent saturated aliphatic radical. The term Cₙ means the alkylene group has “n” carbon atoms. For example, C₁₋₆alkylene refers to an alkylene group having a number of carbon atoms encompassing the entire range, as well as all subgroups, as previously described for “alkyl” groups.

As used herein, “polyoxyalkylene” refers to a bivalent alkyl ether having oxyalkylene repeat units. The term Cₙ means the polyoxyalkylene group has “n” carbon atoms. For example, C₂₋₆polyoxyalkylene refers to an polyoxyalkylene group having a number of carbon atoms encompassing the entire range, as well as all subgroups, as previously described for “alkyl” groups. Examples of polyoxyalkene groups include polyethylene glycol moieties and polypropylene glycol moieties.

As used herein, the term “heteroaryl” refers to a cyclic aromatic ring having five to twelve total ring atoms (e.g., a monocyclic aromatic ring with 5-6 total ring atoms), and containing one to three heteroatoms selected from nitrogen, oxygen, and sulfur atom in the aromatic ring. Unless otherwise indicated, a heteroaryl group can be unsubstituted or substituted with one or more, and in particular one to four, substituents selected from, for example, halo, alkyl, alkenyl, OCF₃, NO₂, CN, NC, OH, alkoxy, amino, CO₂H, CO₂alkyl, aryl, and heteroaryl. In some cases, the heteroaryl group is substituted with one or more of alkyl and alkoxy groups. Heteroaryl groups can be isolated (e.g., pyridyl) or fused to another heteroaryl group (e.g., purinyl), a cycloalkyl group (e.g., tetrahydroquinolinyl), a heterocycloalkyl group (e.g., dihydronaphthyridinyl), and/or an aryl group (e.g., benzothiazolyl and quinolyl). Examples of heteroaryl groups include, but are not limited to, thienyl, furyl, pyridyl, pyrrolyl, oxazolyl, quinolyl, thiophenyl, isoquinolyl, indolyl, triazinyl, triazolyl, isothiazolyl, isoxazolyl, imidazolyl, benzothiazolyl, pyrazinyl, pyrimidinyl, thiazolyl, and thiadiazolyl. When a heteroaryl group is fused to another heteroaryl group, then each ring can contain five or six total ring atoms and one to three heteroatoms in its aromatic ring.

A “substituted” functional group (e.g., a substituted alkyl, alkyleneyl, cycloalkyl, aryl, or heteroaryl refers to an alkyl, alkyleneyl, cycloalkyl, aryl, or heteroaryl) is a functional, group having at least one hydrogen radical that is substituted with a non-hydrogen radical (i.e., a substitutent). Examples of non-hydrogen radicals (or substituents) include, but are not limited to, alkyl, cycloalkyl, alkenyl, cycloalkenyl, alkynyl, ether, aryl, heteroaryl, heterocycloalkyl, hydroxyl, oxy (or oxo), alkoxyl, ester, thioester, acyl, carboxyl, cyano, nitro, amino, sulfhydryl, and halo. When a substituted alkyl group includes more than one non-hydrogen radical, the substituents can be bound to the same carbon or two or more different carbon atoms.

### Pharmaceutically Acceptable Salts

The compounds described herein can exist in free form, or where appropriate, as salts. Those salts that are pharmaceutically acceptable are of particular interest since they are useful in administering the compounds described herein for medical purposes. Salts that are not pharmaceutically acceptable are useful in manufacturing processes, for isolation and purification purposes, and in some cases, for use in separating stereoisomeric forms of the compounds of the disclosure or intermediates thereof.

As used herein, the term “pharmaceutically acceptable salt” refers to salts of a compound which are, within the scope of sound medical judgment, suitable for use in contact with the tissues of humans and lower animals without undue side effects, such as, toxicity, irritation, allergic response and the like, and are commensurate with a reasonable benefit/risk ratio.

Pharmaceutically acceptable salts are well known in the art. For example, S. M. Berge et al., describe pharmaceutically acceptable salts in detail in J. Pharmaceutical Sciences, 1977, 66, 1-19, which is incorporated herein by reference. Pharmaceutically acceptable salts of the compounds described herein include those derived from suitable inorganic and organic acids and bases. These salts can be prepared in situ during the final isolation and purification of the compounds.

Where the compound described herein contains a basic group, or a sufficiently basic bioisostere, acid addition salts can be prepared by 1) reacting the purified compound in its free-base form with a suitable organic or inorganic acid and 2) isolating the salt thus formed. In practice, acid addition salts might be a more convenient form for use and use of the salt amounts to use of the free basic form.

Examples of pharmaceutically acceptable, non-toxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, glycolate, gluconate, glycolate, hemisulfate, heptanoate, hexanoate, hydrochloride, hydrobromide, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, palmoate, pectinate, persulfate, 3-phenylpropionate, phosphate, picrate, pivalate, propionate, salicylate, stearate, succinate, sulfate, tartrate, thiocyanate, p-toluenesulfonate, undecanoate, valerate salts, and the like.

Where the compound described herein contains a carboxyl group or a sufficiently acidic bioisostere, base addition salts can be prepared by 1) reacting the purified compound in its acid form with a suitable organic or inorganic base and 2) isolating the salt thus formed. In practice, use of the base addition salt might be more convenient and use of the salt form inherently amounts to use of the free acid form. Salts derived from appropriate bases include alkali metal (e.g., sodium, lithium, and potassium), alkaline earth metal (e.g., magnesium and calcium), ammonium and N⁺(C₁₋₄alkyl)₄ salts. This disclosure also envisions the quaternization of any basic nitrogen-containing groups of the compounds disclosed herein. Water or oil-soluble or dispersible products may be obtained by such quaternization.

Basic addition salts include pharmaceutically acceptable metal and amine salts. Suitable metal salts include the sodium, potassium, calcium, barium, zinc, magnesium, and aluminum. The sodium and potassium salts are usually preferred. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate. Suitable inorganic base addition salts are prepared from metal bases which include sodium hydride, sodium hydroxide, potassium hydroxide, calcium hydroxide, aluminum hydroxide, lithium hydroxide, magnesium hydroxide, zinc hydroxide and the like. Suitable amine base addition salts are prepared from amines which are frequently used in medicinal chemistry because of their low toxicity and acceptability for medical use. Ammonia, ethylenediamine, N-methyl-glucamine, lysine, arginine, omithine, choline, N,N′-dibenzylethylenediamine, chloroprocaine, dietanolamine, procaine, N-benzylphenethylamine, diethylamine, piperazine, tris(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, dicyclohexylamine and the like.

Other acids and bases, although not in themselves pharmaceutically acceptable, may be employed in the preparation of salts useful as intermediates in obtaining the compounds described herein and their pharmaceutically acceptable acid or base addition salts.

It should be understood that a compound disclosed herein can be present as a mixture/combination of different pharmaceutically acceptable salts. Also contemplated are mixtures/combinations of compounds in free form and pharmaceutically acceptable salts.

### Pharmaceutical Formulations

Also provided herein are pharmaceutical formulations that include an effective amount of compounds of the disclosure and one or more pharmaceutically acceptable excipients. As used herein, the term “formulation” is used interchangeable with “composition.”

An “effective amount” includes a “therapeutically effective amount” and a “prophylactically effective amount.” The term “therapeutically effective amount” refers to an amount effective in treating and/or ameliorating a disease or condition in a subject. The term “prophylactically effective amount” refers to an amount effective in preventing and/or substantially lessening the chances of a disease or condition in a subject. As used herein, the terms “patient” and “subject” may be used interchangeably and mean animals, such as dogs, cats, cows, horses, and sheep (i.e., non-human animals) and humans. Particular patients or subjects are mammals (e.g., humans). The terms “patient” and “subject” include males and females.

As used herein, the term “excipient” means any pharmaceutically acceptable additive, carrier, diluent, adjuvant, or other ingredient, other than the active pharmaceutical ingredient (API), suitably selected with respect to the intended form of administration, and consistent with conventional pharmaceutical practices.

The compounds of the disclosure can be administered alone or as part of a pharmaceutically acceptable composition or formulation. In addition, the compounds can be administered all at once, as for example, by a bolus injection, multiple times, e.g. by a series of tablets, or delivered substantially uniformly over a period of time, as for example, using transdermal delivery. It is also noted that the dose of the compound can be varied over time.

The compounds disclosed herein and other pharmaceutically active compounds, if desired, can be administered to a subject or patient by any suitable route, e.g. orally, topically, rectally, parenterally, (for example, subcutaneous injections, intravenous, intramuscular, intrasternal, and intrathecal injection or infusion techniques), or as a buccal, inhalation, or nasal spray. The administration can be to provide a systemic effect (e.g. eneteral or parenteral). All methods that can be used by those skilled in the art to administer a pharmaceutically active agent are contemplated. In some embodiments, the disclosed formulations can be administered orally or topically.

Suitable oral compositions or formulations in accordance with the disclosure include without limitation tablets, troches, lozenges, aqueous or oily suspensions, dispersible powders or granules, emulsion, hard or soft capsules, syrups or elixirs. Compositions or formulations suitable for oral use may be prepared according to any method known to the art for the manufacture of pharmaceutical compositions.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, com, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in microencapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

The pharmaceutical compositions and formulations described herein may also be administered topically or transdermally, especially when the target of treatment includes areas or organs readily accessible by topical application, including diseases of the eye, the skin, or the lower intestinal tract. Suitable topical formulations are readily prepared for each of these areas or organs. Topical application for the lower intestinal tract, e.g., can be effected in a rectal suppository formulation or in a suitable enema formulation. Dosage forms for topical or transdermal administration of a compound described herein include ointments, pastes, creams, lotions, gels, powders, solutions, sprays, inhalants, suppositories, or patches.

For topical applications, the pharmaceutical compositions may be formulated in a suitable ointment, cream, lotion, or gel, containing the active component suspended or dissolved in one or more carriers, and any needed preservatives or buffers as may be required. Carriers for topical administration of the compounds of this disclosure include, but are not limited to, mineral oil, liquid petrolatum, white petrolatum, propylene glycol, polyoxyethylene, polyoxypropylene compound, emulsifying wax and water. Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2 octyldodecanol, benzyl alcohol and water.

Ophthalmic formulation, eardrops, and eye drops are also contemplated as being within the scope of this disclosure. Additionally, the present disclosure contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound described herein, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are specifically suppositories which can be prepared by mixing the compounds described herein with suitable non-irritating excipients or carriers such as cocoa butter, polyethylene glycol or a suppository wax which are solid at ambient temperature but liquid at body temperature and therefore melt in the rectum or vaginal cavity and release the active compound.

Sterile injectable forms of the compositions described herein may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

The pharmaceutical compositions may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

The compounds for use in the methods of the disclosure can be formulated in unit dosage form. The term “unit dosage form” refers to physically discrete units suitable as unitary dosage for subjects undergoing treatment, with each unit containing a predetermined quantity of active material calculated to produce the desired therapeutic effect, optionally in association with a suitable pharmaceutical carrier. The unit dosage form can be for a single daily dose or one of multiple daily doses (e.g., about 1 to 4 or more times per day). When multiple daily doses are used, the unit dosage form can be the same or different for each dose.

The compounds of the disclosure can be administered to a subject or patient at dosage levels in the range of about 0.1 to about 3,000 mg per day. For a normal adult human having a body weight of about 70 kg, a dosage in the range of about 0.01 to about 100 mg per kilogram body weight is typically sufficient. The specific dosage and dosage range that will be used can potentially depend on a number of factors, including the requirements of the subject or patient, the severity of the condition or disease being treated, and the pharmacological activity of the compound being administered. The determination of dosage ranges and optimal dosages for a particular subject or patient is within the ordinary skill in the art.

### Methods of Treatment

The compounds disclosed herein (e.g., the compounds of Formula (I) or a salt thereof) can function as prodrugs of tapinarof, which is a small molecule that can bind to various biological targets. The prodrugs disclosed herein advantageously can deliver tapinarof directly to a tissue of interest, e.g., to the small and/or large intestine, without significant absorption or metabolism before the compounds reach the intended target tissue. Accordingly, disclosed herein is a method of delivering tapinarof (compound a) to the intestine (e.g., small and/or large intestine) of a subject comprising administering to the subject a prodrug of tapinarof (e.g., compound or salt disclosed herein, such as a compound of Formula (I), or a formulation thereof. In some embodiments, the intestine is the small intestine. In some embodiments, the intestine is the large intestine. In some embodiments, compound a is released from the prodrug or salt in the large intestine.

The compounds disclosed herein, for example, can modulate the aryl hydrocarbon receptor (AHR) pathway, e.g., by modulating AHR in a cell. AHR is a ligand-activated transcription factor that has been implicated in a variety of conditions, including by modulating the immune system during steady state and during infection and inflammation. The AHR pathway has been recognized for its role in the pathogenesis of inflammatory skin diseases such as atopic dermatitis, psoriasis, and vitiligo; intestinal pathologies, such as inflammatory bowel disorder, necrotizing enterocolitis, and other autoimmune diseases, and for colorectal cancer; and in diseases and disorders of the bones and joints, including rheumatoid arthritis; as well as diseases of the eye, such as age-related macular degeneration; and diseases of the lung, such as lung fibrosis, chronic obstructive pulmonary disease.

Thus, the disclosure provides a method of modulating the aryl hydrocarbon receptor (AHR) in a cell comprising contacting the cell with a therapeutically effective amount of a compound or salt disclosed herein (such as a compound of Formula (I)), or a formulation thereof, in an amount effective to modulate the AHR.

In some cases, the contacting occurs in vitro. In some embodiments, the contacting occurs in vivo. In some cases, the contacting comprises administering to a subject in need thereof. As used herein, the terms “patient” and “subject” may be used interchangeably and mean animals, such as dogs, cats, cows, horses, and sheep (i.e., non-human animals) and humans. Particular patients are mammals (e.g., humans). In some cases, the subject suffers from a disease or disorder of the gastrointestinal tract, skin, lung, eye, or bone joints.

Another aspect of the disclosure provides a method of treating a disease or disorder in a subject, comprising administering to the subject a therapeutically effective amount of a compound or salt disclosed herein (such as a compound of Formula (I)), or a formulation thereof. In some embodiments, the terms “treating”, “treat” or “treatment” and the like can include preventative (e.g., prophylactic) and palliative treatment. In some embodiments, the disease or disorder is a disease or disorder of the gastrointestinal tract, skin, lung, eye, or bone joints.

In some embodiments, the disease or disorder of the gastrointestinal tract is selected from the group consisting of colitis, inflammatory bowel disease, Crohn's disease, celiac disease, necrotizing enterocolitis, irritable bowel syndrome, chronic idiopathic constipation, traveler's diarrhea, and colorectal cancer. In various embodiments, the disease or disorder of the gastrointestinal tract is selected from the group consisting of colitis, inflammatory bowel disease, Crohn's disease, celiac disease, necrotizing enterocolitis, and irritable bowel syndrome. In some embodiments, the disease or disorder of the skin is selected atopic dermatitis, psoriasis, and vitiligo. In some embodiments, the disease or disorder of bone joints is selected from the group consisting of osteoporosis, rheumatoid arthritis, and bone cancer.

Another aspect of the disclosure provides the use of a compound or salt disclosed herein (such as a compound of Formula (I)), or a formulation comprising a compound or salt disclosed herein (such as a compound of Formula (I) in the treatment of a disease or disorder of the gastrointestinal tract, skin, or bone joints.

Also contemplated is the use of a compound or salt disclosed herein (such as a compound of Formula (I)), or a formulation comprising a compound or salt disclosed herein (such as a compound of Formula (I) for the manufacture of a medicament in the treatment of a disease or disorder of the gastrointestinal tract, skin, or bone joints.

Use of a compound of Formula (I) as disclosed herein, or a pharmaceutically acceptable salt thereof to treat a disease or disorder of the gastrointestinal tract, skin, or bone joints in a subject also is contemplated. Further, the use of a compound of Formula (I) as disclosed herein, or a pharmaceutically acceptable salt thereof, also is contemplated. Furthermore, use of a compound of Formula (I) as disclosed herein, or a pharmaceutically acceptable salt in the preparation of a medicament for use in treating the aforementioned conditions also are contemplated.

In some embodiments, the compound of Formula (I) as disclosed herein, or pharmaceutically acceptable salt thereof, can be administered in combination with another therapeutic agent to treat a disease or disorder, such a disease or disorder of the gastrointestinal tract, skin, eye, lung, or bone joints. Thus, in any of the methods disclosed herein, treatment of a disease or disorder of the gastrointestinal tract, skin, or bone joints includes co-administration of the compound of Formula (I), or a pharmaceutically acceptable salt thereof in combination with another therapeutic.

In jurisdictions that forbid the patenting of methods that are practiced on the human body, the meaning of “administering” of a composition to a human subject or patient shall be restricted to prescribing a controlled substance that a human subject or patient will self-administer by any technique (e.g., orally, inhalation, topical application, injection, insertion, etc.). The broadest reasonable interpretation that is consistent with laws or regulations defining patentable subject matter is intended. In jurisdictions that do not forbid the patenting of methods that are practiced on the human body, the “administering” of compositions includes both methods practiced on the human body and also the foregoing activities.

### Synthesis of the Compounds of the Disclosure

The compounds of the disclosure can be synthesized by any method known in the art. For example, the compounds of Formula (I) can be synthesized according to Scheme 1.

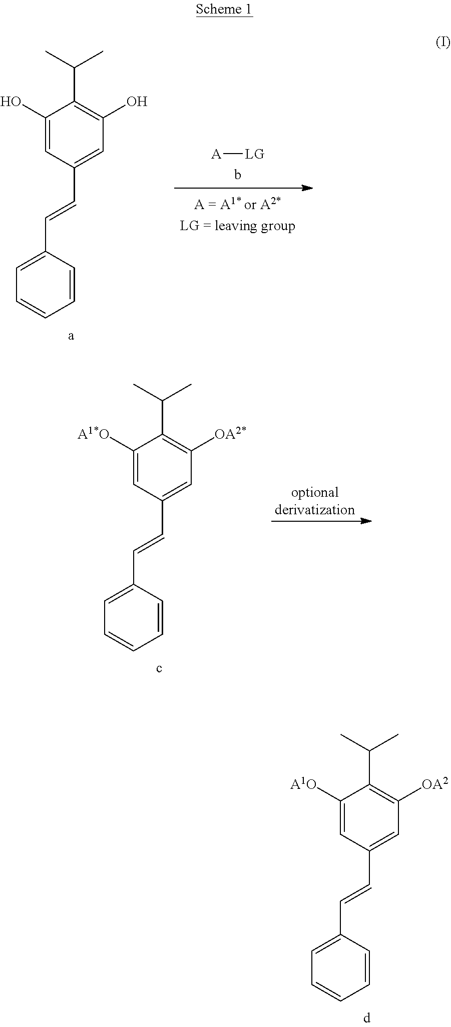

As used herein, LG refers to any suitable moiety known in the art to be displaceable by a nucleophile. Examples of leaving groups include, but are not limited to, halides (e.g., chloride, bromide, iodide), sulfonates (e.g., tosylate, mesylate, triflate), sulfides (e.g,. SCH₃), a carboxylic acid coupling reagent derivative (e.g., N-hydroxsuccinimide, N-hydroxybenzotriazole), an activated carboxylic acid (e.g., acyl chloride), or an acidic hydroxyl group activated via Mitsunobu condition. Nucleophiles are known in the art and include amines, alcohols, and thiols.

In some embodiments, compounds of Formula (I) having structure d can be synthesized using the procedure shown in Scheme 1. Reaction of 2-isopropyl-5-[(E)-2-phenylethenyl]benzene-1,3-diol a with a nucleophilic A derivative compound b produces substituted 2-isopropyl-5-[(E)-2-phenylethenyl]benzene compounds having structure c. Optional subsequent derivatization gives compounds as described herein, i.e., compounds of Formula I having structure d. Appropriate derivatization reactions can be selected based on the nature of substituents A¹* and A²*.

The coupling of compounds a and b can be catalyzed or facilitated by appropriate reagents selected based on the precise nature of compounds a and b. For example, when compound b is a phosphoroyl compound (i.e., when A¹ and/or A² is a phosphate group), the coupling of compound a and a phosphoroyl compound b can be catalyzed by a base e.g., triethylamine. Occasionally, the coupling reaction may not require a catalyst, or the solvent for the reaction can be the catalyst, e.g., when compound b is a sulfur trioxide pyridine complex and the solvent is pyridine (i.e., when A¹ and/or A² is a sulfate group).

Compounds a and b can be purchased commercially or prepared by a variety of methods from commercially-available starting materials. Optional derivatization reactions to transform compounds having structure c into compounds having structure d can be selected based on the nature of the substituents A¹* and A²* in compound c, and the functionality desired in compound d. For example, when A¹* and A²* is a 4-oxobutanoic acid, the terminal acid functionality can be further derivatized by methods known in the art to form a variety of functional groups. For example, the acid moiety can be conjugated to an a-cyclodextrin. Derivatization of acid can be effected via known methods such as carbodiimide chemistry, or through the use of catalytic reagents such as HATU and the like, according to the nature of the derivatization reaction as disclosed herein.

### EXAMPLES

The following examples are provided for illustration and are not intended to limit the scope of the invention.

### Example 1. Preparation of (E)-4-(3-hydroxy-2-isopropyl-5-styrylphenoxy)-4-oxobutanoic acid (Compound 1)

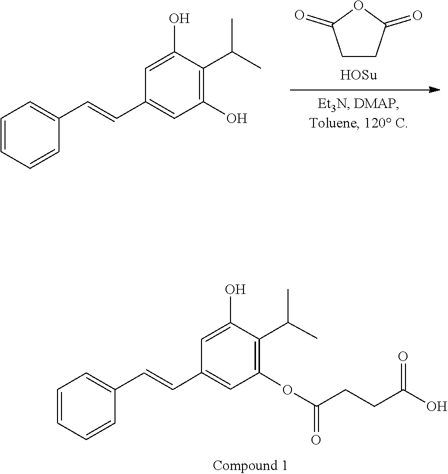 [SMILES: CC(C)c1c(O)cc(/C=C/c2ccccc2)cc1O ; O=C1CCC(=O)O1 ; CC(C)c1c(O)cc(/C=C/c2ccccc2)cc1OC(=O)CCC(=O)O]

To a solution of 2-isopropyl-5-[(E)-2-phenylethenyl]benzene-1,3-diol (1000.0 mg, 3.9 mmol) in toluene (20 ml) was added oxolane −2,5-dione (1950.0 mg, 19.5 mmol), 1-hydroxypyrrolidine-2,5-dione (897.0 mg, 7.8 mmol), triethylamine [789.0 mg, 7.8 mmol] and dimethylaminopyridine (DMAP) (143.0 mg, 1.17 mmol). The reaction mixture was stirred at 100° C. for 16 h under N₂ atmosphere. The residue was extracted with EtOAc (200 mL×3), the organic layer was washed with brine, dried over Na₂SO₄, filtered and concentrated to crude product which was purified by flash chromatography (PE/EtOAc=100/0 to 40/60) to afford compound 1 (500.0 mg, yield: 36.0%). ¹H NMR (400 MHz, MeOD, ppm): 7.51 (d, J=7.4 Hz, 2H), 7.34 (t, J=7.6 Hz, 2H), 7.23 (t, J=7.4 Hz, 1H), 7.09-6.96 (m, 2H), 6.83 (d, J=1.5 Hz, 1H), 6.69 (d, J=1.5 Hz, 1H), 3.27-3.14 (m, J=14.1, 7.1 Hz, 1H), 2.94-2.85 (m, 2H), 2.78-2.68 (m, 2H), 1.28 (d, J=7.1 Hz, 6H). LCMS (ESI) calcd for C₂₁H₂₂O₅ [M+H]⁺ m/z 355.2, found 355.2. HPLC: 254 nm (99.4%), 214 nm (97.4%).

### Example 2. Preparation of 5-{3-[(4-carboxybutanoyl)oxy]-2-isopropyl-5-[(E)-2-phenylethenyl]phenoxy}-5-oxopentanoic acid (Compound 4)

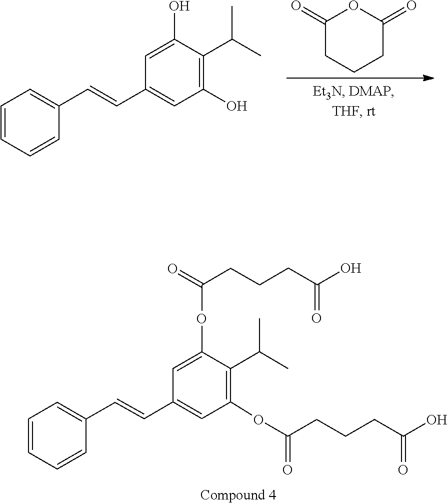 [SMILES: CC(C)c1c(O)cc(/C=C/c2ccccc2)cc1O ; O=C1CCCC(=O)O1 ; CC(C)c1c(OC(=O)CCCC(=O)O)cc(/C=C/c2ccccc2)cc1OC(=O)CCCC(=O)O]

To a solution of 2-isopropyl-5-[(E)-2-phenylethenyl]benzene-1,3-diol (200.0 mg, 0.786 mmol) and oxane-2,6-dione (269.0 mg, 2.36 mmol) in THF (4 ml) was added triethylamine (159.0 mg, 1.57 mmol) and DMAP (19.0 mg, 0.158 mmol) under N₂ atmosphere. The reaction mixture was stirred at room temperature for 16 h. The residue was extracted with EtOAc (200 mL), the organic layer was washed with brine, dried over Na₂SO₄, filtered and concentrated to get crude product which was purified by flash chromatography (PE/EtOAc=100/0 to 40/60) to afford compound 4 (46.0 mg, yield: 12.1%). ¹H NMR (400 MHz, CD₃OD, ppm): 7.55 (d, J=7.7 Hz, 2H), 7.35 (t, J=7.4 Hz, 2H), 7.26 (t, J=7.1 Hz, 1H), 7.21-7.06 (m, 4H), 3.16-3.03 (m, 1H), 2.75 (t, J=7.2 Hz, 4H), 2.48 (t, J=7.1 Hz, 4H), 2.07-2.02 (m, 4H), 1.24 (d, J=6.9 Hz, 6H). LCMS (ESI) calcd for C₂₇H₃₀O₈ [M+Na]⁺ m/z 505.2, found 505.2. HPLC: 254 nm (99.4%), 214 nm (97.4%).

### Example 3: Preparation of (E)-3-hydroxy-2-isopropyl-5-styrylphenyl dihydrogen phosphate (Compound 25)

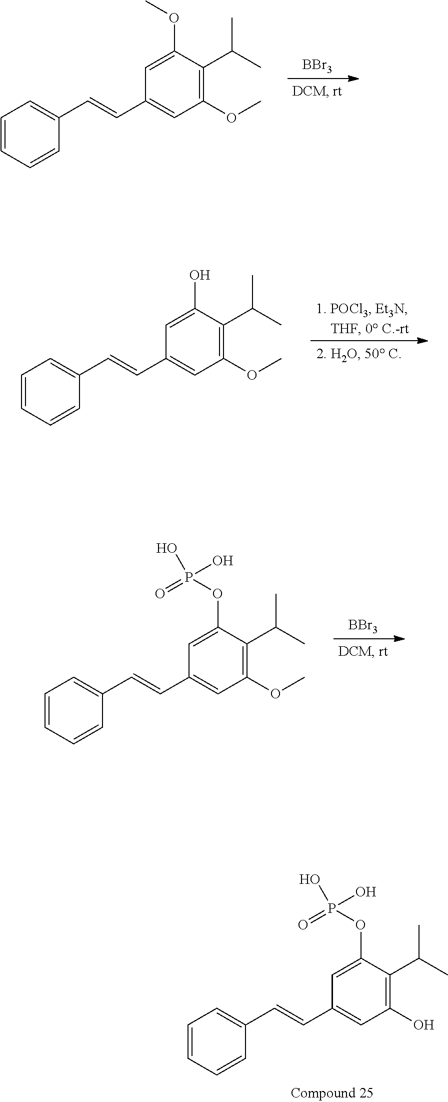

To a solution of 2-isopropyl-1,3-dimethoxy-5-[(E)-2-phenylethenyl]benzene (877.0 mg, 3.10 mmol) in dichloromethane (DCM) was added tribromoborane (3 mL, 1 M in DCM) dropwise at −78° C. The reaction mixture was stirred at 25° C. for 12 h. The residue was quenched by water, concentrated in vacuo and the crude product was purified by flash chromatography (PE/EtOAc=100/0 to 90/10) to afford (E)-2-isopropyl-3-methoxy-5-styrylphenol (310.0 mg, yield: 37.3%). ¹H NMR (400 MHz, MeOD, ppm): 7.52 (d, J=7.6 Hz, 2H), 7.34 (t, J=7.6 Hz, 2H), 7.23 (t, J=7.3 Hz, 1H), 7.10-6.98 (m, 2H), 6.62 (d, J=13.5 Hz, 2H), 3.83 (s, 3H), 3.60-3.50 (m, 1H), 1.29 (d, J=7.1 Hz, 6H). LCMS (ESI) calcd for C₁₈H₂₀O₂ [M+H]+ m/z 269.2, found 269.2.

To a solution of 2-isopropyl-5-[(E)-2-phenylethenyl]benzene-1,3-diol (250.0 mg, 0.93 mmol) in anhydrous tetrahydrofuran (THF, 5 ml) was added phosphoroyl trichloride (428.5 mg, 2.79 mmol) and triethylamine (565.6 mg, 5.59 mmol) dropwise at 0° C. The reaction was stirred at 25° C. for 3 h and water (2 mL) was added then stirred at 50° C. for 2 h. After completion, the solvent was removed by vacuum to crude product, which was purified by prep-HPLC [H₂O (0.1% FA)/MeCN=100/0 to 20/80) to afford (E)-2-isopropyl-3-methoxy-5-styrylphenyl dihydrogen phosphate (18.0 mg, yield: 5.5%). ¹H NMR (400 MHz, MeOD, ppm): 7.54 (d, J=7.6 Hz, 2H), 7.34 (t, J=7.6 Hz, 2H), 7.27-7.17 (m, 2H), 7.15-7.06 (m, 2H), 6.93 (s, 1H), 3.88 (s, 3H), 3.62-3.53 (m, 1H), 1.31 (d, J=7.1 Hz, 6H). LCMS (ESI) calcd for C₁₈H₂₁O₅P [M−H]− m/z 349.2, found 349.2.

To a solution of 2-isopropyl-1,3-dimethoxy-5-[(E)-2-phenylethenyl]benzene (200.0 mg, 0.57 mmol) in DCM was added tribromoborane (0.7 mL, 1 M in DCM) dropwise at −78° C. The reaction mixture was stirred at 25° C. for 12 h. The LCMS showed the desired MS was detected. The residue was quenched by water and removed by vacuum to crude product which was purified by prep-HPLC [H₂O (0.1%FA)/MeCN=100/0 to 50/50) to afford compound 25 (30.0 mg, yield: 16.7%). ¹H NMR (400 MHz, MeOD, ppm): 7.52-7.46 (m, 2H), 7.31 (t, J=7.8 Hz, 2H), 7.07-6.94 (m, 1H), 7.07-6.94 (m, 3H), 6.73 (s, 1H), 3.57-3.45 (m, 1H), 1.33 (d, J=7.1 Hz, 6H). LCMS (ESI) calcd for C₁₇H₁₉O₅P [M+H]+ m/z 333.2, found 333.2. HPLC: 254 nm (99.4%), 214 nm 93.3%).

### Example 4. Preparation of (E)-2-isopropyl-5-styryl-1,3-phenylene bis(dihydrogen phosphate) (Compound 28)

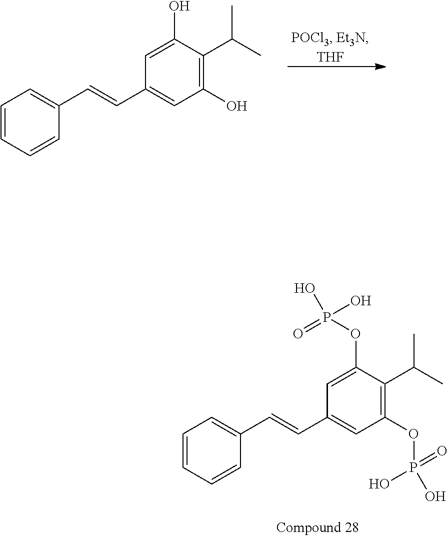

To a solution of 2-isopropyl-5-[(E)-2-phenylethenyl]benzene-1,3-diol (200.0 mg, 0.78 mmol) in anhydrous THF (5 ml) was added phosphoroyl trichloride (396.0 mg, 2.59 mmol) and triethylamine (744.1 mg, 4.68 mmol) dropwise at 0° C. The reaction mixture was stirred at 25° C. for 3 h. After completion, the residue was quenched by ice water, concentrated in vacuo and the crude product was purified by prep-HPLC [H₂O (0.1%FA)/MeCN=100/0 to 30/70) to afford compound 28 (7.44 mg, yield: 2.2%). ¹H NMR (400 MHz, MeOD, ppm): 7.51 (d, J=7.6 Hz, 2H), 7.41 (s, 2H), 7.32 (t, J=7.5 Hz, 2H), 7.21 (dd, J=13.6, 6.2 Hz, 1H), 7.15 (s, 1H), 7.06 (d, J=16.4 Hz, 1H), 3.71-3.55 (m, 1H), 1.37 (d, J=7.0 Hz, 6H). LCMS (ESI) calcd for C₁₇H₂₀O₈P₂ [M−H]− m/z 413.2, found 413.2. HPLC: 254 nm (96.3%), 214 nm (98.1%).

### Example 5: Preparation of (E)-3-hydroxy-2-isopropyl-5-styrylphenyl hydrogen sulfate (Compound 31)

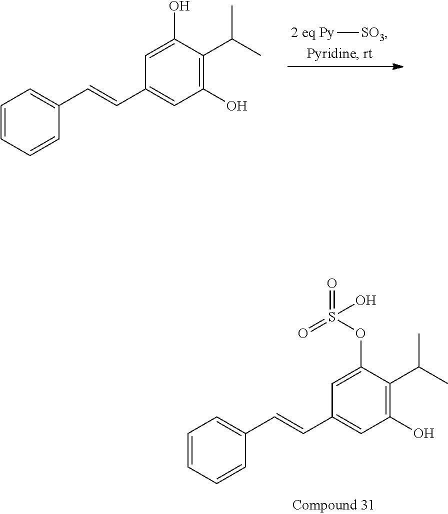

To a solution of 2-isopropyl-5-[(E)-2-phenylethenyl]benzene-1,3-diol (300.0 mg, 1.18 mmol) in pyridine was added SO₃·Py (375.0 mg, 2.36 mmol). The reaction mixture was stirred 25° C. for 3 h. The solvent was evaporated under reduced pressure and the crude product was purified by prep-HPLC [H₂O (0.1% NH₃—H₂O)/MeCN=100/0 to 20/80] to afford compound 31 (76.0 mg, yield: 19.2%) as a yellow solid. ¹H NMR (400 MHz, MeOD, ppm): 7.52 (d, J=7.4 Hz, 2H), 7.34 (t, J=7.6 Hz, 2H), 7.27-7.18 (m, 2H), 7.05 (q, J=16.3 Hz, 2H), 6.77 (d, J=1.6 Hz, 1H), 3.67-3.57 (m, 1H), 1.36 (d, J=7.1 Hz, 6H). LCMS (ESI) calcd for C₁₇H₁₈O₅S [M−H]− m/z 332.9, found 332.9. HPLC: 254 nm (97.8%), 214 nm (95.5%).

### Example 6: Preparation of (E)-2-isopropyl-5-styryl-1,3-phenylene bis(hydrogen sulfate) (Compound 34)

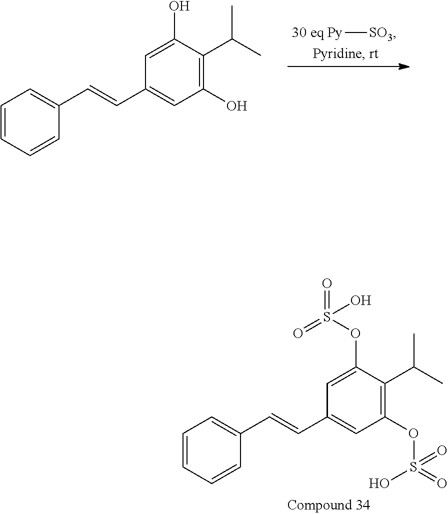

To a solution of 2-isopropyl-5-[(E)-2-phenylethenyl]benzene-1,3-diol (300.0 mg, 1.18 mmol) in pyridine was added SO₃·Py (5611.7 mg, 35.27 mmol). The reaction mixture was stirred 25°° C. for 3 h. The solvent was evaporated under reduced pressure and the crude product was purified by prep-HPLC [H₂O (0.1% FA)/MeCN=100/0 to 50/50] to afford compound 34 (50.0 mg, yield: 10.2%) as a yellow solid. ¹H NMR (400 MHz, MeOD, ppm): 7.60 (s, 2H), 7.54 (d, J=7.4 Hz, 2H), 7.34 (t, J=7.6 Hz, 2H), 7.26-7.18 (m, 1H), 7.16-7.06 (m, 2H), 3.74-3.62 (m, 1H), 1.40 (d, J=7.1 Hz, 6H). LCMS (ESI) calcd for C₁₇H₁₈O₈S₂ [M−H]− m/z 413.1, found 413.1. HPLC:254 nm (97.8%), 214 nm (97.8%).

Example 7. Preparation of 1-[31,32,33,34,35,36,37,38,39,40,41,42-dodecahydroxy-10,15,20,25,30-pentakis(hydroxymethyl)-2,4,7,9,12,14,17,19,22,24,27,29-dodecaoxaheptacyclo[26.2.2.2\hat{ }{3,6}.2\hat{ }{8,11}.2\hat{ }{13,16}.2\hat{ }{18,21}.2\hat{ }{23,26}]dotetracontan-5-yl]methyl 3-hydroxy-2isopropyl- 5-[(E)-2-phenylethenyl]phenyl butanedioate (Compound 36)

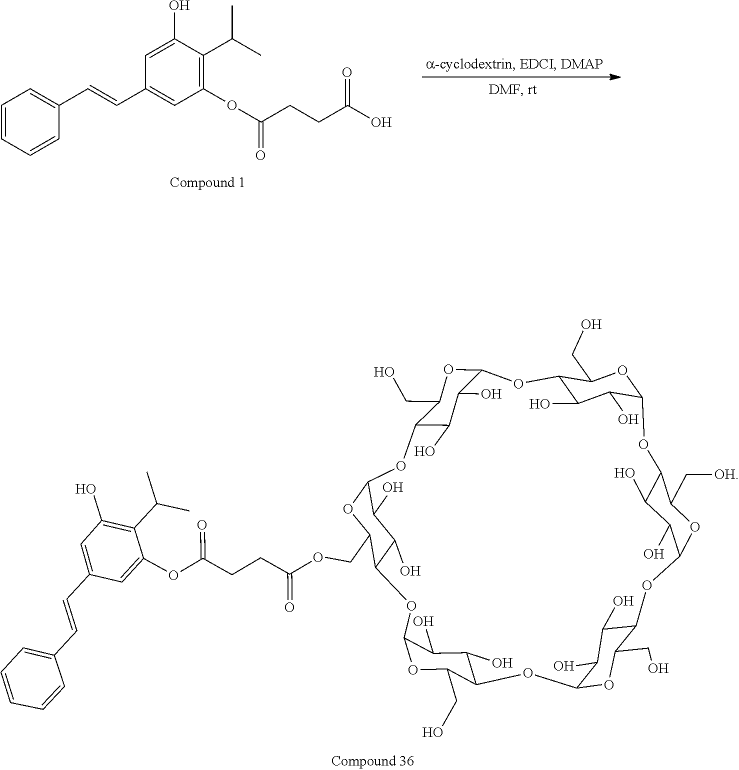 [SMILES: CC(C)c1c(O)cc(/C=C/c2ccccc2)cc1OC(=O)CCC(=O)O ; CC(C)c1c(O)cc(/C=C/c2ccccc2)cc1OC(=O)CCC(=O)OCC1OC2OC3C(CO)OC(OC4C(CO)OC(OC5C(CO)OC(OC6C(CO)OC(OC7C(CO)OC(OC1C(O)C2O)C(O)C7O)C(O)C6O)C(O)C5O)C(O)C4O)C(O)C3O]

To a stirred solution of compound 1 (500.0 mg, 1.4 mmol) in dimethylformamide (DMF, 10 mL) was added a-cyclodextrin (1647.0 mg, 1.69 mmol), 1-ethyl-3-carbodiimide (EDCl, 541.0 mg, 2.8 mmol) and DMAP (51.7 mg, 0.42 mmol) at room temperature. The reaction mixture was stirred at room temperature for 16 h under nitrogen. The LCMS showed the desired MS was detected. The solvent was removed by vacuum to crude product which was purified by reversed phase chromatography [H₂O (0.1% FA)/MeCN=100/0 to 30/70) to afford compound 36 (23.0 mg, yield: 1.2%). ¹H NMR (400 MHz, DMSO-d₆, ppm): 9.69 (br, 1H), 7.59 (d, J=7.6 Hz, 2H), 7.36 (t, J=7.6 Hz, 2H), 7.26 (t, J=7.3 Hz, 1H), 7.07 (q, J=16.3 Hz, 2H), 6.87 (s, 1H), 6.72 (d, J=7.1 Hz, 1H), 5.70-5.35 (m, 12H), 4.84-4.76 (m, 5H), 4.58-4.44 (m, 4H), 4.35-4.27 (m, 2H), 3.78 (t, J=8.7 Hz, 6H), 3.72-3.56 (m, 18H), 3.48-3.39 (m, 6H), 3.30-3.22 (m, 7H), 2.91-2.82 (m, 2H), 2.78-2.69 (m, 2H), 1.20 (d, J=6.9 Hz, 6H). LCMS (ESI) calcd for C₅₇H₈₀O₁₄ [M+H]⁺ m/z=1309.5, found 1309.5. HPLC: 254 nm (95.9%), 214 nm (95.1%).

### Example 8: In Vitro Stability Screening

The stability of the compounds of Formula (I) was assessed by exposing representative compounds to simulated gastric fluids. In particular, representative compounds of the disclosure were incubated in one or more of the simulated gastric fluids known to those skilled in the art for up to 12 hours at a temperature in range of about 20° C. to about 37° C. The amount of prodrug remaining after the incubation period was determined by LCMS.

### Example 9: Ex Vivo Release Studies

The ability of the compounds of Formula (I) to release the active compound in the small or large intestine was assessed by introducing representative compounds of the disclosure to the contents of either a rodent small intestine or a rodent large intestine for up to 12 hours at a temperature in range of about 20° C. to about 37° C. The amount of active drug released after the incubation period was determined by LCMS.

### Example 10: Pharmacokinetics Studies

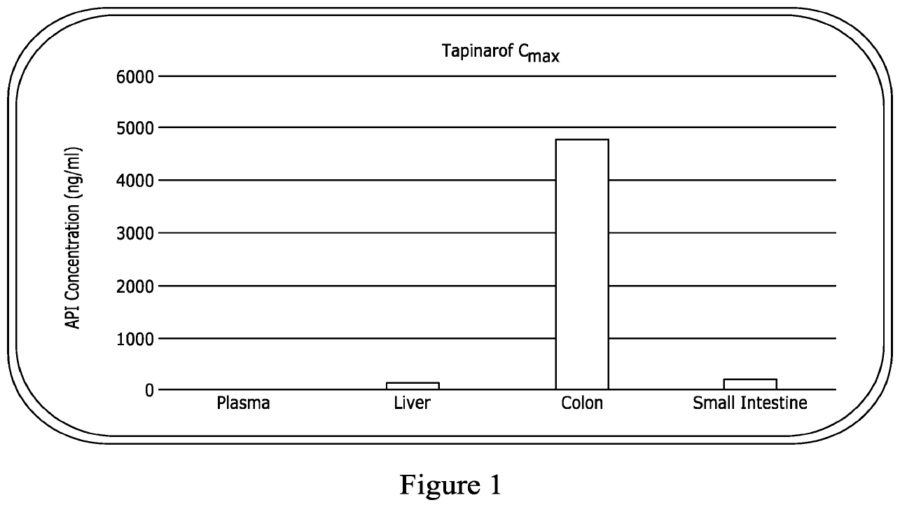

Pharmacokinetics studies of the compounds disclosed herein were conducted in different species of experimental animals follow standard protocols familiar to those skilled in the art. For example, illustrated below is a pharmacokinetics study of Compound 31 in Balb/6 mice at a dose equivalent to 30 mpk Tapinarof. Six groups of mice (male, 3 in each group) were randomized and treated orally with prodrug molecule. Blood, colon, small intestine, and liver samples were taken at 0.5, 1, 2, 4, 8, 24 h time points. Tapinarof concentration was analyzed and Cmax distribution across the tissues are shown in FIG. 1.

### Example 11-In vivo efficacy:

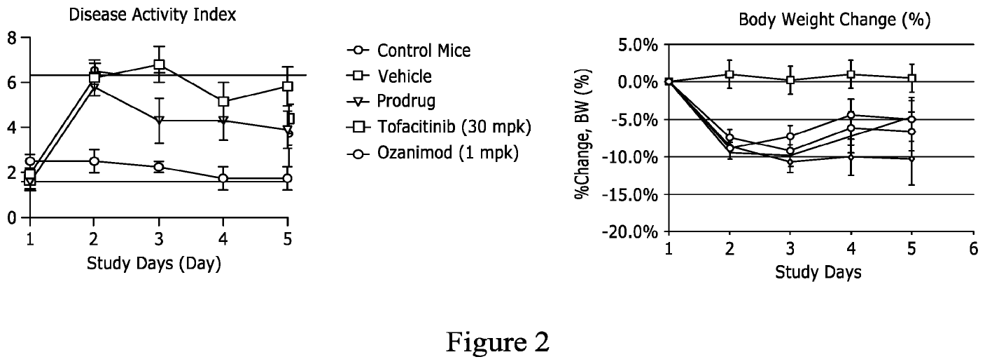

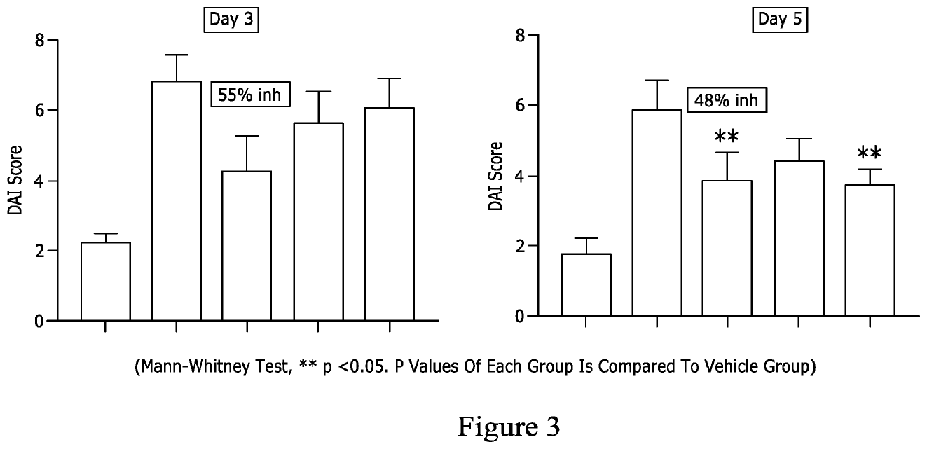

Compound 31 was used in a TNBS-induced Inflammatory Bowel Disease (IBD) model in Balb/c mice (8 to 10-week-old, male). Mice were randomized based on their body weight and the date of randomization was denoted as Day 1. Under anesthesia, mice were held by the tail. A 3.5-French catheter was gently inserted through the anus into the colon for ˜4 cm and 100 μl of 1% (wt/vol) TNBS solution was injected into the lumen of the colon slowly. The catheter was then withdrawn from the colon slowly and the animal was held by the tail to maintain the head down position for another 1 min to ensure the TNBS solution remains completely in the colon. The animals were then placed back into the cage. Upon randomization, Compound 31 and reference compounds (vehicle, tofacitinib, or ozanimod) were orally administered to the mice from Day 1 to Day 5. Body weight was measured daily from Day 1 to Day 5. In addition, animals were checked daily for morbidity and mortality. The body weight changes, the stool consistency and blood in stool, the Disease Activity Index (DAI) were scored daily after randomization from Day 1 to Day 5. Results for disease activity index (DAI) and body weight changes are outlined in FIG. 2, where Compound 31 (labeled “prodrug” in Figure) was dosed at equivalent of 30 mpk tapinarof. FIG. 3 shows the DAI scores for the different reference compounds compared to Compound 31 and against control mice-from left to right control, vehicle, Compound 31 (30 mpk tapinarof), tofacitinib (30 mpk), and ozanimod (1 mpk).

The foregoing description is given for clearness of understanding only, and no unnecessary limitations should be understood therefrom, as modifications within the scope of the invention may be apparent to those having ordinary skill in the art.

Throughout this specification and the claims which follow, unless the context requires otherwise, the word “comprise” and variations such as “comprises” and “comprising” will be understood to imply the inclusion of a stated integer or step or group of integers or steps but not the exclusion of any other integer or step or group of integers or steps.

Throughout the specification, where compositions are described as including components or materials, it is contemplated that the compositions can also consist essentially of, or consist of, any combination of the recited components or materials, unless described otherwise. Likewise, where methods are described as including particular steps, it is contemplated that the methods can also consist essentially of, or consist of, any combination of the recited steps, unless described otherwise. The invention illustratively disclosed herein suitably may be practiced in the absence of any element or step which is not specifically disclosed herein.

The practice of a method disclosed herein, and individual steps thereof, can be performed manually and/or with the aid of or automation provided by electronic equipment. Although processes have been described with reference to particular embodiments, a person of ordinary skill in the art will readily appreciate that other ways of performing the acts associated with the methods may be used. For example, the order of various of the steps may be changed without departing from the scope or spirit of the method, unless described otherwise. In addition, some of the individual steps can be combined, omitted, or further subdivided into additional steps.

All patents, publications and references cited herein are hereby fully incorporated by reference. In case of conflict between the present disclosure and incorporated patents, publications and references, the present disclosure should control.

We claim:

1. A compound having a structure of Formula (I): 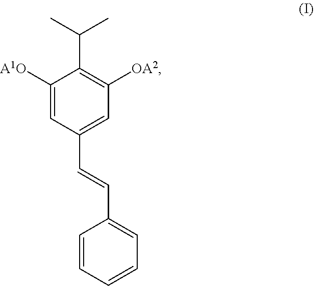
or a pharmaceutically acceptable salt thereof, wherein: 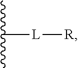
each of A¹ and A² independently is H, C₁₋₆alkyl, or and at least one of A¹ and A² is 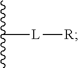
each L independently is a bond, 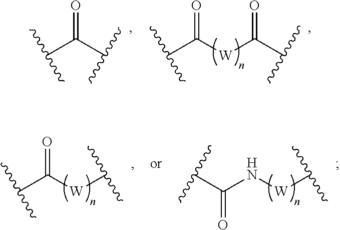
W is 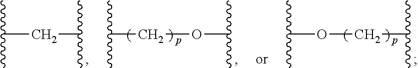
n is an integer from 1-6;
p is 2 or 3; and
each R independently is a polar group capable of forming at least two hydrogen bonds.

2. The compound or salt of claim 1, wherein one of A¹ and A² is H or C₁₋₆alkyl and the other of A¹ and A² is

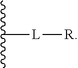

3. The compound or salt of claim 2, wherein the one of A¹ and A² is H or CH₃.

4. The compound or salt of claim 1, wherein each of A¹ and A² is

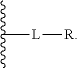

5. The compound or salt of any one of claims 1-4, wherein each R independently is selected from the group consisting of 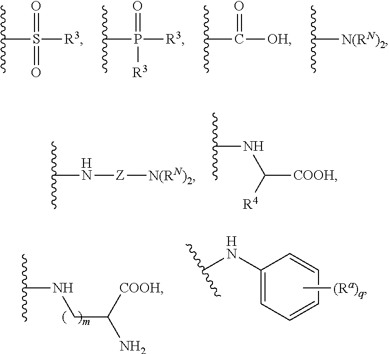
and a saccharide;
wherein:
m is 1, 2, 3, 4, 5, or 6;
q is 2, 3, 4, or 5;
Z is C₂₋₆alkylene or C₂₋₆polyoxyalkene;
each R^{a} independently is 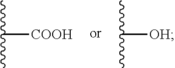
each R^{N} independently is H or CH₃;
each R³ independently is OH or NH₂;
each R⁴ independently is H, C₁₋₆alkyl, or 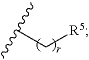
wherein:
r is an integer from 1-6;
R⁵ is selected from the group consisting of 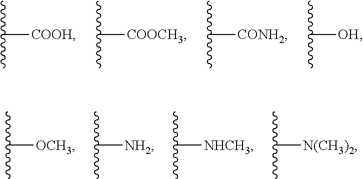
heteroaryl comprising 5 or 6 total ring atoms and 1, 2, or 3 heteroatoms selected from N, O, and S, 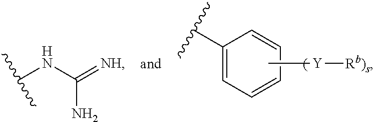
wherein s is 1, 2, 3, 4, or 5; Y is absent or C₁₋₂alkylene; and R^{b} is selected from the group consisting of

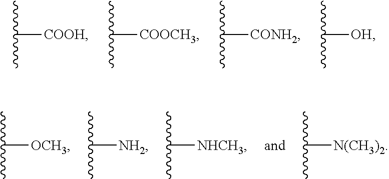

6. The compound or salt of any one of claims 1-5, wherein at least one L is a bond.

7. The compound or salt of any one of claims 1 and 4-6, wherein each L is a bond.

8. The compound or salt of claim 6 or 7, wherein R selected from the group consisting of

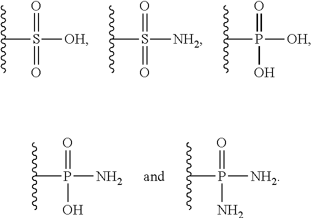

9. The compound or salt of claim 8, wherein: (i) each R is 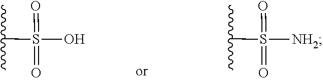 [SMILES: *S(=O)(=O)O ; *S(N)(=O)=O]
or (ii) each R is

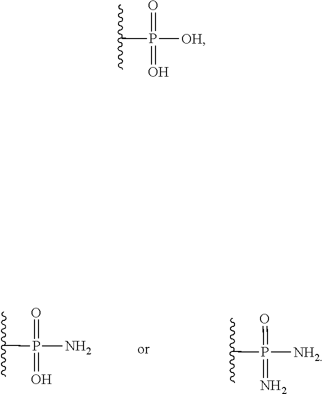

10. The compound or salt of claim 9, wherein: (i) each R is 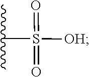 [SMILES: *S(=O)(=O)O]
or (ii) each R is

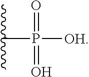

11. The compound or salt of any one of claims 1-5, wherein at least one L is

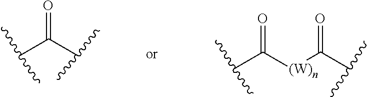

12. The compound or salt of claim 11, wherein n is 2, 3, or 4.

13. The compound or salt of claim 11 or 12, wherein W is

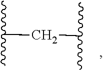 [SMILES: *C*]

14. The compound or salt of claim 11 or 12, wherein W is 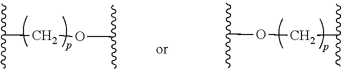
and p is 2.

15. The compound or salt of any one of claims 11-14, wherein L is not a bond and each R independently is 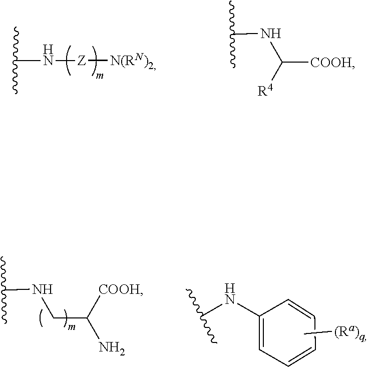
or a saccharide.

16. The compound or salt of claim 15, wherein at least one R is 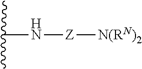
and each R^{N} is H.

17. The compound or salt of claim 16, wherein at least one R is

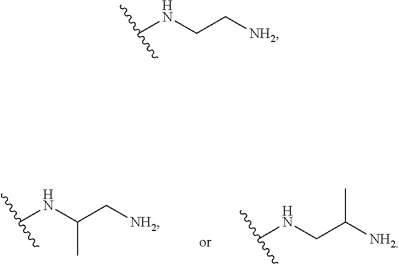

18. The compound or salt of claim 15, wherein at least one R is 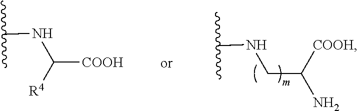
R⁴ is H or CH₃, and m is 1, 2, or 3.

19. The compound or salt of claim 15, wherein at least one R is 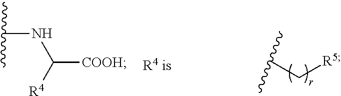
r is 1, 2, or 3; and R⁵ is

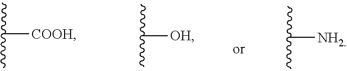

20. The compound or salt of claim 18 or 19, wherein at least one R is

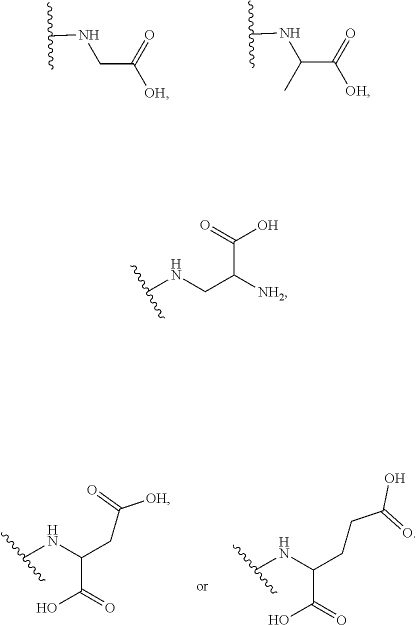

21. The compound or salt of claim 15, wherein at least one R is 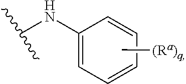
q is 2 or 3; and each R^{a} independently is

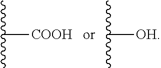

22. The compound or salt of claim 21, wherein at least one R is

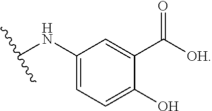 [SMILES: *Nc1ccc(O)c(C(=O)O)c1]

23. The compound or salt of claim 15, wherein at least one R is a saccharide.

24. The compound or salt of claim 23, wherein the saccharide is an amino saccharide, a monosaccharide, a disaccharide, or an oligosaccharide.

25. The compound or salt of claim 24, wherein the saccharide is an oligosaccharide comprising 3-10 monosaccharide units.

26. The compound or salt of claim 24 or 25, wherein the oligosaccharide comprises α-1,4 glycosidic bonds.

27. The compound or salt of any one of claims 24-26, wherein the oligosaccharide is a cyclic oligosaccharide having 5-8 pyranose units.

28. The compound or salt of claim 27, wherein the pyranose units comprise glucose.

29. The compound or salt of any one of claims 25-28, wherein the oligosaccharide comprises a cyclodextrin.

30. The compound or salt of claim 29, wherein at least one R is

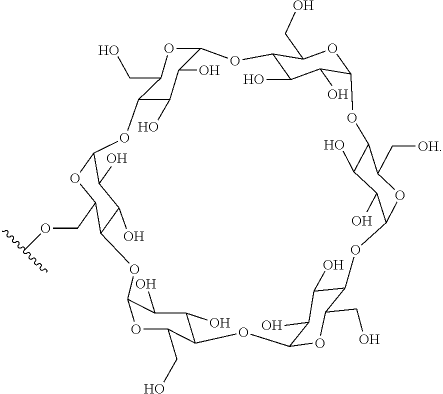 [SMILES: *OCC1OC2OC3C(CO)OC(OC4C(CO)OC(OC5C(CO)OC(OC6C(CO)OC(OC7C(CO)OC(OC1C(O)C2O)C(O)C7O)C(O)C6O)C(O)C5O)C(O)C4O)C(O)C3O]

31. The compound or salt of any one of claims 1-5, wherein at least one L is

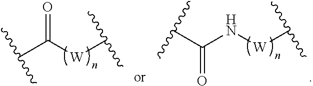

32. The compound or salt of claim 31, wherein n is 2, 3, or 4.

33. The compound or salt of claim 31 or 32, wherein W is

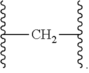 [SMILES: *C*]

34. The compound or salt of any one of claims 31-33, wherein at least one L is

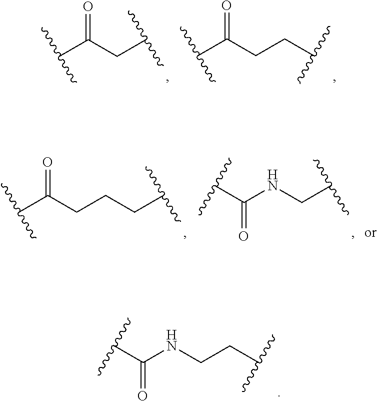 [SMILES: *CC(*)=O ; *CCC(*)=O ; *CCCC(*)=O ; *CNC(*)=O ; *CCNC(*)=O]

35. The compound or salt of claim 31 or 32, wherein W is 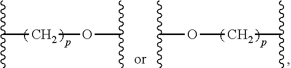
and p is 2.

36. The compound or salt of claim 35, wherein at least one L is

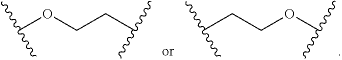 [SMILES: *CCO* ; *CCO*]

37. The compound or salt of any one of claims 31-36, wherein at least one R is

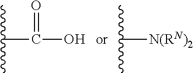

38. The compound or salt of claim 37, wherein at least one R is

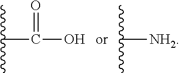

39. The compound or salt of claim 38, where each R independently is

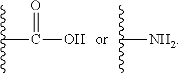

40. The compound or salt of claim any one of claims 1-5 wherein at least one of A¹ and A² is selected from the group consisting of

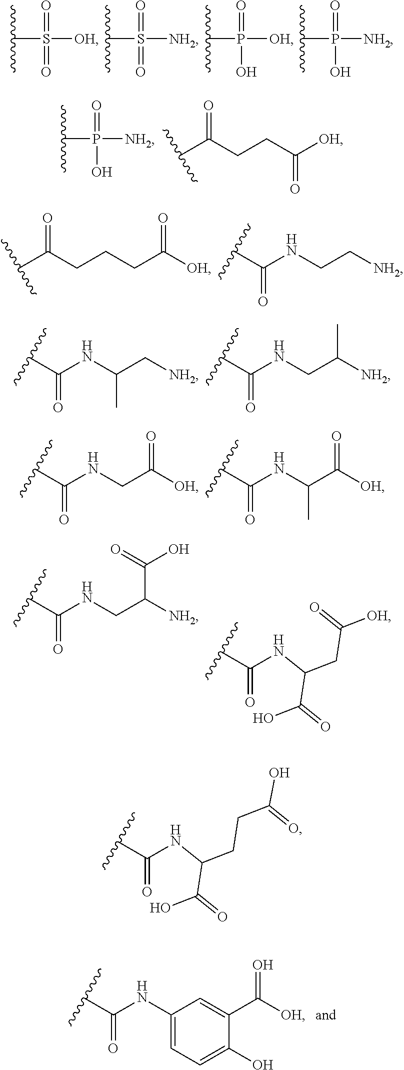

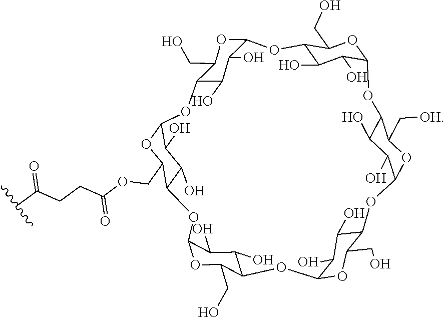 [SMILES: *C(=O)CCC(=O)OCC1OC2OC3C(CO)OC(OC4C(CO)OC(OC5C(CO)OC(OC6C(CO)OC(OC7C(CO)OC(OC1C(O)C2O)C(O)C7O)C(O)C6O)C(O)C5O)C(O)C4O)C(O)C3O]

41. The compound or salt of any one of claims 4-40, wherein A¹ and A² are the same.

42. The compound or salt of any one of claims 4-40 wherein A¹ and A² are different.

43. The compound of claim 1 having a structure selected from the group consisting of: 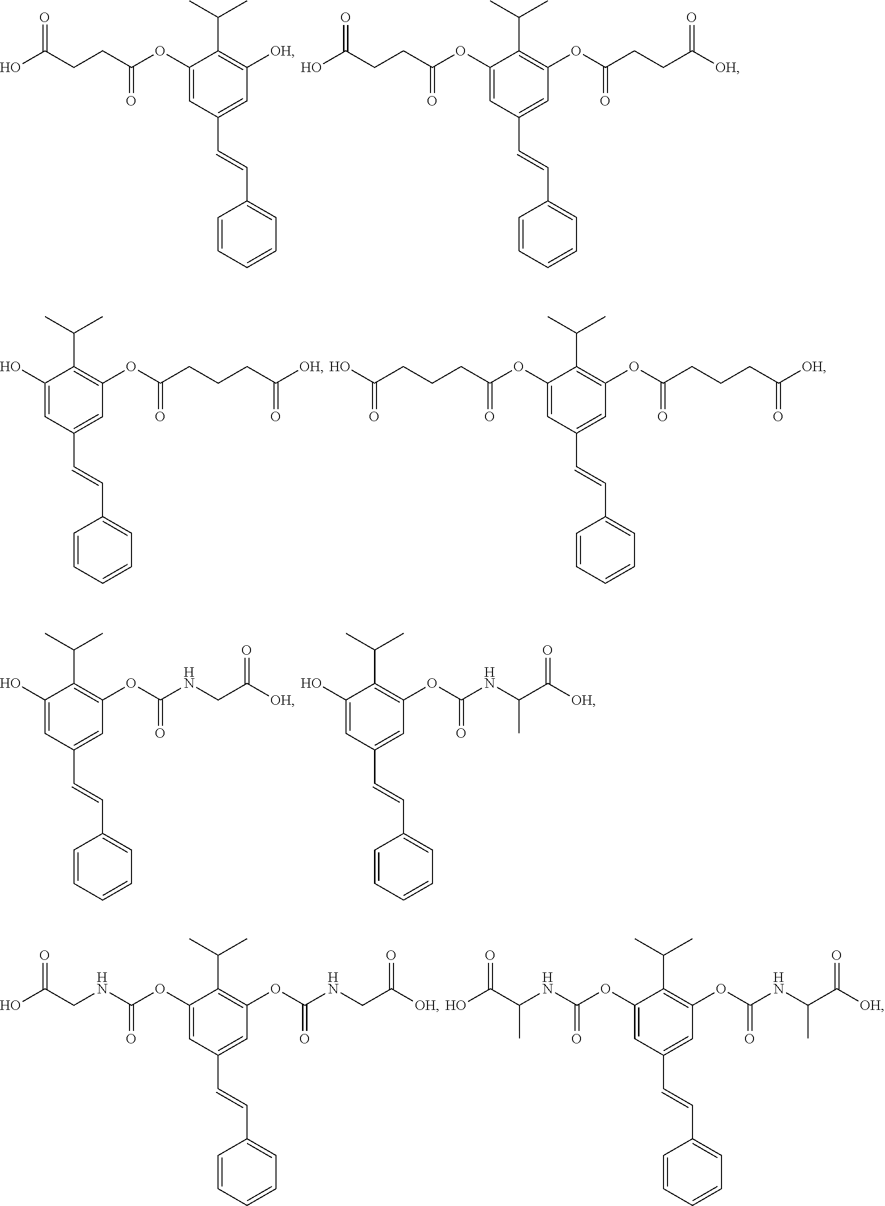 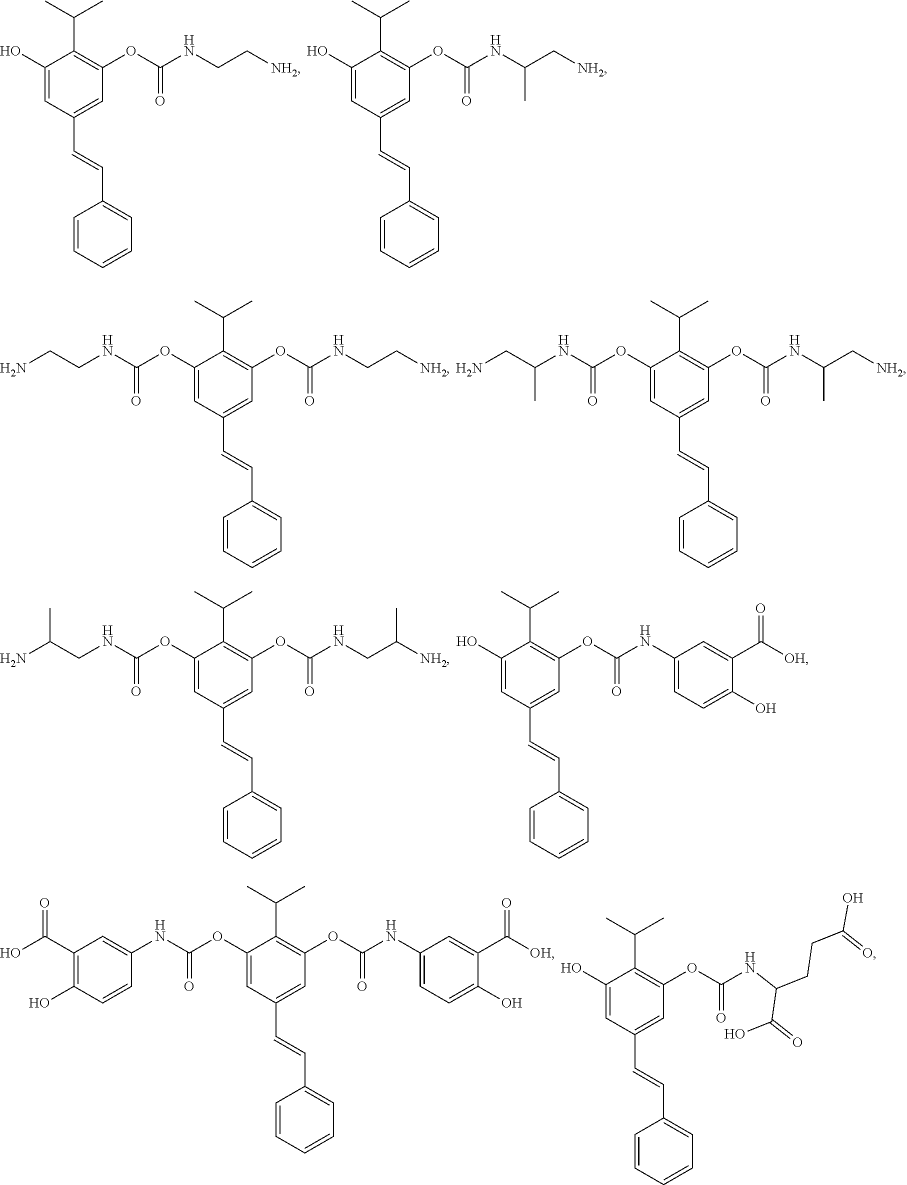 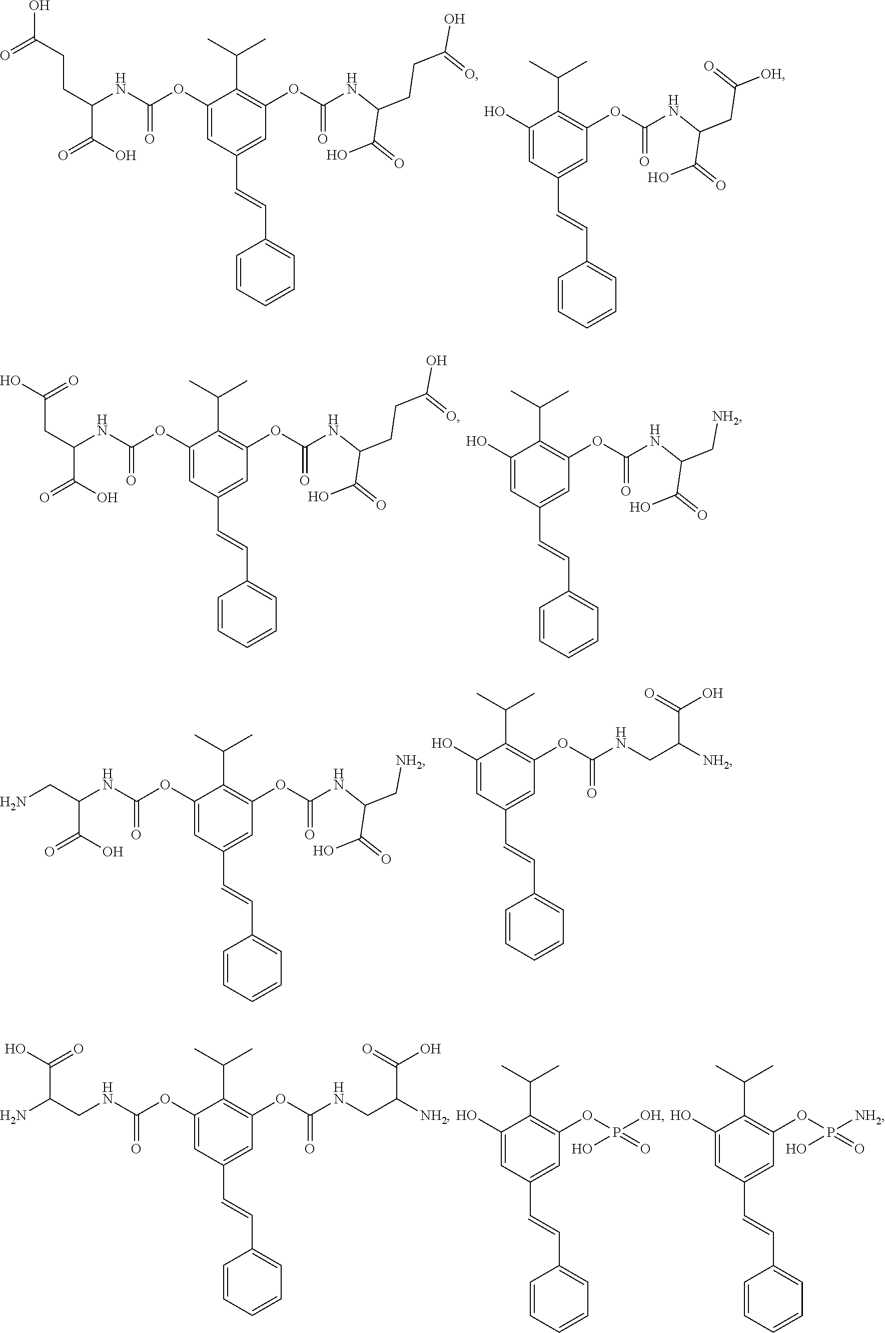 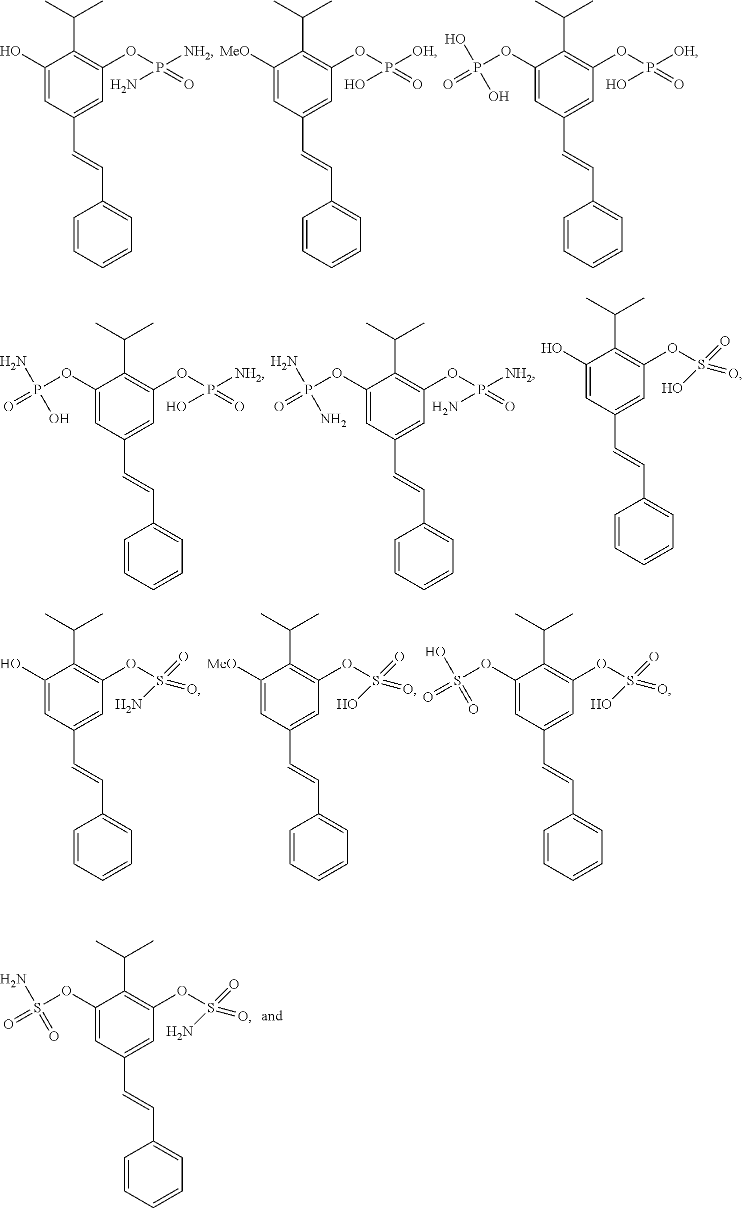 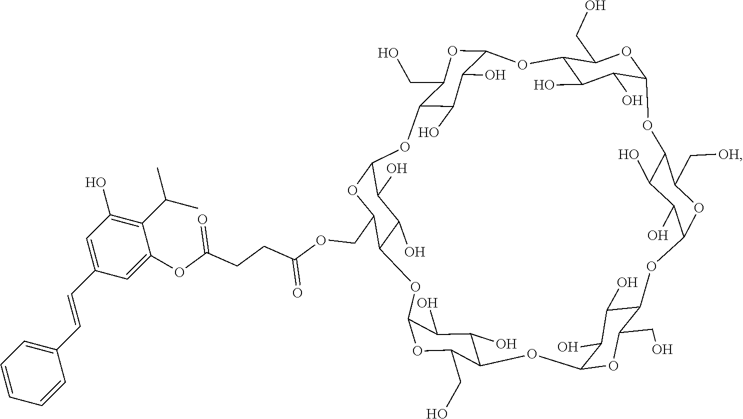 [SMILES: CC(C)c1c(O)cc(/C=C/c2ccccc2)cc1OC(=O)CCC(=O)OCC1OC2OC3C(CO)OC(OC4C(CO)OC(OC5C(CO)OC(OC6C(CO)OC(OC7C(CO)OC(OC1C(O)C2O)C(O)C7O)C(O)C6O)C(O)C5O)C(O)C4O)C(O)C3O]
or a pharmaceutically acceptable suit thereof.

44. The compound or salt of any one of claims 1-43, wherein the compound or salt is optically pure.

45. The compound or salt of any one of claims 1-44, wherein the compound or salt comprises one or more deuterium atones.

46. A pharmaceutical formulation comprising the compound or salt of any one of claims 1-45 and a pharmaceuticaly acceptable excipient

47. The formulation of claim 46 as an oral formulation.

48. The formulation of claim 46 as a topical formulation.

49. A method of delivering 2-isopropyl-5-(E)-2-phenylethenyl]benzene-1,3-diol (compound a): 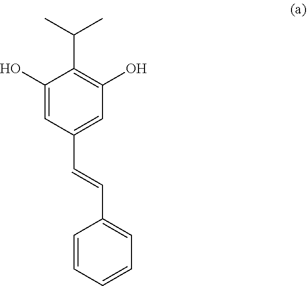 [SMILES: CC(C)c1c(O)cc(/C=C/c2ccccc2)cc1O]
to the intestine of a subject comprising administering to the subject a compound or salt of any one of claims 1-45 or a formulation of claim 46 or 47.

50. The method of claim 49, wherein compound a is released from the prodrug or salt in the intestine.

51. The method of claim 49 or 50, wherein the intestine is the small intestine.

52. The method of claim 49 or 50, wherein the intestine is the large intestine.

53. A method of modulating the aryl hydrocarbon receptor (AHR) in a cell comprising contacting the cell with a therapeutically effective amount of a compound or salt of any one of claims 1-45 or a formulation of any one of claims 46-48 in an amount effective to modulate the AHR.

54. The method of claim 53, wherein the contacting occurs in vivo.

55. The method of claim 53 or 54, wherein the contacting comprises administering to a subject in need thereof.

56. The method of claim 55, wherein the subject suffers from a disease or disorder of the gastrointestinal tract, skin, or bone joints.

57. A method of treating a disease or disorder in a subject, comprising administering to the subject a therapeutically effective amount of a compound or salt of any one of 1-45 or a formulation of any one of claims 46-48.

58. The method of claim 57, wherein the disease or disorder is a disease or disorder of the gastrointestinal tract, skin, eye, lung, or bone joints.

59. The method of claim 57 or 58, wherein the disease or disorder of the gastrointestinal tract is selected from the group consisting of colitis, inflammatory bowel disease, Crohn's disease, celiac disease, necrotizing enterocolitis, irritable bowel syndrome, chronic idiopathic constipation, traveler's diarrhea, and colorectal cancer.

60. The method of claim 57 or 58, wherein the disease or disorder of the skin is selected from the group consisting of atopic dermatitis, psoriasis, and vitiligo.

61. The method of claim 57 or 58, wherein the disease or disorder of the eye is age-related macular degeneration.

62. The method of claim 57 or 58, wherein the disease or disorder of the lung is lung fibrosis or chronic obstructive pulmonary disease.

63. The method of claim 57 or 58, wherein the disease or disorder of the bone joints is selected from the group consisting of osteoporosis, rheumatoid arthritis, and bone cancer.

64. A compound or salt of any one of claims 1-45, or a formulation of any one of claims 46-48 for use in the treatment of a disease or disorder of the gastrointestinal tract, skin, eye, lung or bone joints.

65. Use of a compound or salt of any one of claims 1-45, or the formulation of any one of claims 46-48 for the manufacture of a medicament for use in the treatment of a disease or disorder of the gastrointestinal tract, skin, eye, lung, or bone joints.

66. A method of preparing the compound or salt of any one of claims 1-44, comprising admixing 2-isopropyl-5-[(E)-2-phenylethenyl]benzene-1,3-diol (compound a): 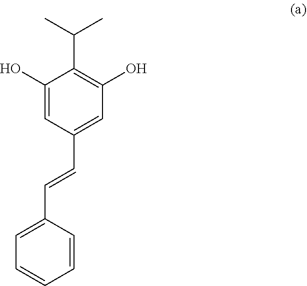 [SMILES: CC(C)c1c(O)cc(/C=C/c2ccccc2)cc1O]
with A¹-LG, A²-LG, or both; wherein LG is a leaving group, to form a compound of Formula (I) or salt thereof.

67. A prodrug of 2-isopropyl-5-[(E)-2-phenylethenyl]benzene-1,3-diol (compound a):

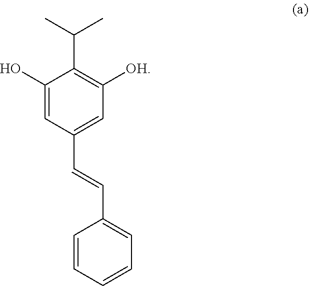 [SMILES: CC(C)c1c(O)cc(/C=C/c2ccccc2)cc1O]

68. A method of delivering 2-isopropyl-5-(E)-2-phenylethenyl|benzene-1,3-diol (compound a): 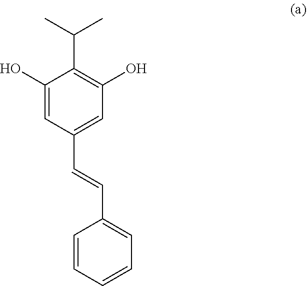 [SMILES: CC(C)c1c(O)cc(/C=C/c2ccccc2)cc1O]
to the intestine of a subject comprising administering to the subject a prodrug of compound (a), or a pharmaceutically acceptable salt thereof.

69. The method of claim 68, wherein compound a is released from the prodrug or salt in the intestine.

70. The method of claim 68 or 69, wherein the intestine is the small intestine.

71. The method of claim 68 or 69, wherein the intestine is the large intestine.
